(19) United States
(12) Patent Application Publication
(10) Pub. No.: US 2025/0090359 A1
(43) Pub. Date: Mar. 20, 2025

(54) BRACE OR SUPPORT WITH ATFL SUPPORT

(71) Applicant: RUBBER CITY BRACING COMPANY LLC, AKRON, NC (US)

(72) Inventors: Dustin Ducharme, Littleton, CO (US); Annunziato Amendola, Durham, NC (US); Bryan Den Hartog, Urbandale, IA (US); David B. Kay, Akron, OH (US); Anthony Perera, Cardiff (GB); Brian Milliff, Kirtland, OH (US); Patrick Brown, Cleveland Heights, OH (US)

(21) Appl. No.: 18/967,264

(22) Filed: Dec. 3, 2024

Related U.S. Application Data
(63) Continuation of application No. 17/509,683, filed on Oct. 25, 2021.
Application No. 17/509,683 is a continuation of application No. 15/349,617, filed on Nov. 11, 2016, now abandoned.
(60) Provisional application No. 62/254,312, filed on Nov. 12, 2015.
(60) Provisional application No. 62/254,320, filed on Nov. 12, 2015.
(60) Provisional application No. 62/254,325, filed on Nov. 12, 2015.
(60) Provisional application No. 62/254,329, filed on Nov. 12, 2015.
(60) Provisional application No. 62/254,342, filed on Nov. 12, 2015.
(60) Provisional application No. 62/355,032, filed on Jun. 27, 2016.
(60) Provisional application No. 62/355,044, filed on Jun. 27, 2016.

(51) Int. Cl. A61F 5/01

(52) U.S. Cl. CPC A61F 5/0111

(57) ABSTRACT

A preferred embodiment of the invention relates to an elastomeric athletic or orthopedic brace and provides support for a joint complex. The preferred embodiment includes an elastomeric sleeve having a distal portion and a proximal portion that surrounds and supports one or more joints and with fenestrations or cut-outs and optional supplemental supports or framework so as to provide an external anatomically configured network that augments the effects of the ligaments. The brace can be used prophylactically or therapeutically.

### FIELD OF THE INVENTION

The invention relates to generally to an elastomeric athletic or orthopedic brace, support that mimics the manner in which the ligaments provide support for a joint complex, and in particular, having an external, adjustable support for additional joint stabilization.

### BACKGROUND OF THE INVENTION

The invention generally provides an athletic or orthopedic brace or support, which in a first embodiment, involves an elastomeric sleeve having fenestrations meaning in this instance, areas of reduced support, which may be openings, or which may include an area of a softer or more yielding material characterized by a lower durometer material. The brace surrounds and supports one or more joints so as to provide an external anatomically configured framework which mimics or augments the effects of the ligaments. In a further embodiment, the brace includes a portion that can be opened and closed such as with adjustable strap members that also can be used to achieve tensioning as needed.

The brace can be used prophylactically (for example, allowing sufficient range of motion to allow the brace to be worn during athletic activities without hindering the athlete, but which acts to support the joint or joint complex and to inhibit potentially harmful motion) or the brace can be used therapeutically (for example, in the aid of healing of a joint or joint complex which has suffered some previous injury). The brace has application in all of the joints, including the shoulder, elbow, wrist, hand, thumb, foot, knee, hip and back and the concepts of the present invention can be applied to each of these joint complexes, but is illustrated specifically with respect to an ankle brace. The brace further comprises embodiments in which 1) the brace is adjustable and includes self-closure mechanisms such as a strap or web of linking members that also act to add support as well as provide for size, fit, or tensioning adjustment; in which 2) the brace includes movement monitoring means molded into or inserted in pockets in the brace or the pockets are provided with additional support or rigid stiffening members; in which 3) the brace includes integral use specific functional stiffening members or protection, such as shin or forearm guards; and in which 4) the brace includes optional additional elastic motion related support members which are directed to specific physiological or kinesthetic purposes.

Joint sprains are a common occurrence and in particular, ankle sprains account for an estimated 2 million injuries per year in the United States, and occur in nearly all types of sporting events, making them the most common sports-related injury. A practical method of decreasing the number and severity of these injuries would clearly be of great benefit since ankle sprains result in a risk of further, and even more severe injury and lasting ankle problems, as well as significant time away from games and practices. To this end, many people use prophylactic bracing or ankle taping as a means to decrease the risk of injury, including people who have suffered in the past from a sprained ankle, or in instances where there may be an increased tendency to injury, such as for joints that are subjected to rigorous use or use in uneven terrain. However, while taping is commonly viewed as effective, it is extremely labor intensive, is good for a single use, and requires an educated application all of which cause it to be very expensive. Thus, the present invention provides an alternative solution, which is easy to put on, is durable, and which is a relatively inexpensive way to achieve a similar or better result.

It is helpful to understand ankle anatomy in order to understand ankle sprains. The ankle (talar) joint has three bones and three lateral groups of stabilizing ligaments. The talus articulates in a hinge fashion with both the tibia and the fibula. The distal tibia and fibula are stabilized by the tibiofibular ligaments (anterior and posterior), also known as the syndesmosis. The thick deltoid ligament supports the medial aspect of the ankle and helps limit eversion. The medial ankle is the site of fewer injuries in the ankle since it is inherently more stable than the lateral ankle. Most ankle sprains are inversion injuries involving either complete or partial tearing of the lateral ligament complex. This complex is composed of three distinct ligaments: the anterior talofibular (ATFL), the calcaneofibular (CFL), and the posterior talofibular (PTFL) which are typically injured in a sequential fashion from anterior to posterior, depending on the severity of the inversion.

Studies have shown that rapid lateral body movement actually accounts for relatively few inversion sprains and further that most ankle sprains occur when landing from a jump, with the foot in an inverted, plantar-flexed position. Several studies support the theory that ankle sprains frequently involve disruption in ankle proprioception that prevents the ankle from protecting itself. Eversion ankle sprains, however, have been found to be the result of outside forces such as contact with another player), rather than the result of inadequate proprioception.

The present invention provides an ankle brace as an alternative to ankle taping. The braces of the prior art have been used instead of traditional taping by many athletes at all levels of competition and offer several advantages: for example, braces are reusable, re-adjustable and can be self-applied. There are estimates that professionally applied taping can run in the tens of thousands of dollars for a professional athlete for a full season of play. Ankle taping is estimated at approximately three times more expensive than bracing over the course of a competitive season.

However, the prior art bracing has disadvantages including the fact that many athletes feel less comfortable or stable when wearing braces than they do when the ankle is taped. Braces also can become worn out, or torn and require frequent replacement, for example, many brace designs use hook and loop fasteners or Velcro™, which has a tendency to relax or slip during use. They also can be considered to be cumbersome or intrusive to the range of motion, and proprioception necessary to compete in some sports. In addition, many athletes prefer the feeling of control that taping presents, although it has been shown that taping can lose its supportive effect after a short period of active use.

Many studies have compared taping versus bracing of the ankle, but it is difficult to control the many variables associated with ankle injuries (including for example, playing surface, shoe wear, individual inherent stability, and intensity of competition on both a team and individual level). Most of these studies have shown that the prior art braces are slightly more effective than taping and that both are better than no support, and further that external ankle stabilization does decrease inversion sprains. The mechanism for this protection is not fully understood. While it would appear that external devices would increase the structural stability of the ankle and make the ankle less susceptible to inversion, studies have shown that there may be additional factors that lead to the beneficial result. It has been determined in one study, for example, that taped participants had improved proprioception both before and after exercise compared with untapped controls. The authors of that study postulated that the traction and/or pressure imparted to the skin of the foot and ankle via taping or bracing provided improved sensory input and thus improved proprioception, resulting in fewer ankle sprains. Another study compared the neuromuscular properties of taped versus un-taped ankles so as to theorize on a measure termed the proprioceptive amplification ratio (PAR), which incorporates neuromuscular properties such as proprioception and degree of mechanical stress. That study found that taping did provide increased ankle protection.

While some concern has been expressed that prolonged taping or bracing of the ankle may result in weakened ankles that are more prone to injury, at least one study has shown that consistent ankle brace use did not change the latency to inversion of the peroneus longus (an important stabilizer of the ankle, particularly against inversion, the most common type of ankle injury).

The present invention provides an answer to the issues of injury related and prophylactic ankle support in the form of an elastomeric ankle brace that provides for directed and anatomically configured support, as well as proprioceptive reinforcement for the brace user. The brace of the present invention provides an increased PAR as compared to the prior art bracing which is a result of the support framework providing stop limited vector directed support in addition to or combination with surface achieved tactile response.

### SUMMARY OF THE INVENTION

The brace is in the form of a sleeve (which can be considered a single unit that spans the joint or a first part on one side of the joint, and a second part on the second side of the joint) and formed from an elastic material in which a more distal portion comprises a loop, which is preferably a continuous loop, which encircles a portion on one side of the joint or joint complex and a more proximal portion that encircles the other side of the joint or joint complex. The brace further includes negative or void areas (i.e., “fenestrations”), such as weakened areas, recesses or apertures that act to re-direct forces through the complementary areas which assume the stresses in response to the existence of the negative area. In further embodiments the sleeve also includes a web or framework of stiffer, more rigid, or less elastic support members that interconnect with each other and between a proximal and distal anchor, each of which encircle the limb. This web or framework acts to augment the natural ligaments. In a way that provides support, but which limits potentially harmful motion.

The ankle brace which illustrates the present invention includes the toot portion and the leg portion which join together at the ankle joint, and is comprised of an interrupted web (in this case meaning a flat, and potentially homogeneous cast or molded sheet) of elastomeric material in which the interruptions or openings together with the material characteristics of the elastomer define the manner in which the brace functions. In particular, the material forming the foot portion and the leg portion are separated by the heel opening and the TFT opening which allow the foot portion and the heel portion to form a joint there between, and to accommodate movement at the ankle joint without unnecessary material or bunching. This is a particular advantage for a soft brace that is worn underneath a shoe, other athletic footwear, or a shin guard. In addition, the combination of the malleoli openings and the two openings at the medial and lateral surfaces of the plantar covering of the foot portion of the brace act so as to provide direction as to resistance of force sustained within the web of material that is defined by the combination of the openings. Thus, the brace of the current invention is designed to allow as much safe freedom of movement to the wearer as possible, but to provide resistance to movement that could be harmful. In particular, the device is intended to inhibit inversion in plantar flexion (and to help stabilize the syndesmotic ligament) so as to avoid “rolling” an ankle. The brace is intended to provide external support tantamount to external ligaments and or fascia, that reinforces in proper places, but which relieves pressure where it is needed. Thus, the device acts in tension and compression to buttress the syndesmotic ligament at the top, and in the cross-configuration to buttress the ATFL (anterior tibiofibular ligament), and the CFL (calcaneal fibular ligament), with a medial web member that buttresses the deltoid ligament. In addition, the elastomeric nature of the brace material, coupled with the form can act to provide energy re-balance to the wearer, where the kinetic energy is re-circulated or re-coiled to the user, while inhibiting potentially dangerous forces applied to the joint complex. The material also provides proprioceptive feed-back to the user and the elasticity and/or stickiness of the material helps to remind the user to maintain tone. It is preferable that the material is “alive” or slightly sticky to the skin of the wearer. A desirable level of stickiness would be the feel of slightly under-cured natural latex, or a material that has been exposed and allowed to dry to a solution of sugar-water, or something less adhesive than a traditional band-aid or a light masking tape. Acceptable values measured according to ASTM, D3330D/D3330M, Test Method F at 90°, for peel adhesion of pressure sensitive tape, would be 0.0005-50 N/100 mm, preferably 0.5-30 N/100 mm, and most preferably 0.2-25 N/100 mm.

In a further embodiment of the invention, additional, and optionally external adjustable struts are provided to provide joint stability against typical directions of ligament strain. Specifically, as relates to the brace of the present invention in use for ankle support, the struts are provided as two additional add-on elastomeric strap members that extend diagonally across the lateral malleoli to provide lines of support in two more or less orthogonal directions extending inferiorly to superiorly and posteriorly to anteriorly respectively. Since these straps can be added to the brace to supplement the brace itself, they can be provided with more or less stretch to provide for more or less support to the joint. Advantageously, the straps have easy attachment means, such as the illustrated puck and grommet mechanism, in which the straps include spaced apart pucks that can be pushed into a retained relationship with the grommet, and that can be popped out of engagement by pulling outward on the extensions.

In an adjustable version, the brace is in the form of a sleeve (which can be considered a single unit) that spans the joint or a first part on one side of the joint which comprises a flat web or band of material that is wrapped around a body on one side of the joint and is closed by closure means which provide for adjustability and for the ability to provide directed tensioning. The sleeve also includes a second part on the second side of the joint and formed from a continuous (i.e. integral) elastic material where a more distal portion comprises a loop, which is preferably a continuous loop (and here it is envisioned that this loop could also be formed by closing a flat web to form a circle), which encircles a portion on one side of the joint or joint complex and a more proximal portion that encircles the other side of the joint or joint complex.

The leg portion of the brace includes a proximal opening that encircles the lower leg sufficiently above the lateral and medial malleoli in order to provide a suitable proximal anchor on the leg of the user. A second distal anchor is joined at the foot opening. This portion of the brace also forms a continuous loop, but advantageously is openable, for example, to the rear, or preferably slightly lateral to the Achilles tendon, for entry into the brace, and also to provide adjustability in this portion of the brace. In a further embodiment, the first and second anchors are stiffer more rigid elements, formed for example of a higher durometer material, (e.g., 85+/−30 and preferably 70+/−10, and most preferably 70+/−5 durometer on the Shore A scale.) The anchors are interconnected by supports or struts in the form of strips, or bands which have a much longer length than width (I.e., more than 5×, and preferably more than 10× but where the width is between ⅛ to ½ inch and the lengths are from ½ to 15 inches depending on whether the length is taken for a single segment, which may be as short as ½ inch or as long as 10 inches extending along a line, or for an aggregate of a number of segments) of the same or similar material. The supports interconnect to form a framework or network of ligament complementary support which aids the joint and inhibits “harmful motion while freely permitting acceptable motion.

Various closure mechanisms can be used at the anchor juncture(s), including straps, bands, webs, and cables having a closure means that mates with a corresponding closure means on the lateral side of the sleeve. These closure means could include buttons, hooks, latches, ratchet mechanisms, post and pin, groove and slide, hook and loop, post and loop, Velcro, cables, and zippers to name a few. The present invention also provides novel mechanisms for closure of a soft and/or elastomeric brace.

The brace further includes negative or void areas, such as weakened areas,), recesses or apertures that act to re-direct forces through the complementary areas which assume the stresses in response to the existence of the negative area. The weakened areas can comprise complimentary webs of softer, more yielding, lower durometer material, e.g., having a durometer of 35+/−10, and preferably 40+/−5, on the Shore A scale. This material may also include perforations, such as pores or holes of 0.0001-0.05 inch diameter, to allow for the evaporation of perspiration. These pores may also affect the softness of the material.

In further embodiments, the distal and proximal anchors are connected structurally by struts or supports which are different than the basic sleeve webbing (i.e., by bands, straps, laces, or cables which are less elastic than the remainder of the brace,) so as to transmit forces directly between them and between the distal and proximal anchors by means of supports which can permit desired motion which is deemed to be within a healthy range of motion, but restrain undesired motion, which would be potentially harmful to a vulnerable joint. The higher stiffness (or lower elasticity or resistance to stretch) can be effected by a number of methods, including a change in material, a change in material characteristics, including cross-linking or durometer which can be caused by the manufacturing method or by the ingredients, or a change in the geometry, including thicker or wider or higher volume of material so as to direct, inhibit or manipulate forces transmitted to the affected joint during use.

Finally, the brace can include pockets for sensors including motion of pressure sensors, including for example, transducers or accelerometers, that can be used for kinetic assessment such as standard gait analysis, or athletic training. Alternatively, these sensors can be integrated or embedded into the brace.

### BRIEF DESCRIPTION OF THE DRAWINGS

FIG. 1 is a side view of the right ankle brace in accordance with the present invention shown on the ankle of a user;

FIG. 2 is a side view of the medial side of the ankle brace of FIG. 1;

FIG. 3 is a side view of the lateral side of the ankle brace of FIG. 1;

FIG. 4 is a view looking down from the top of the leg portion of the ankle brace of FIG. 1;

FIG. 5 is a view looking up from the bottom of the foot portion of the ankle brace of FIG. 1;

FIG. 6 is a view looking forward from the posterior of the foot portion of the ankle brace of FIG. 1;

FIG. 7 is a view looking back from the anterior of the foot portion of the ankle brace of FIG. 1;

FIG. 8 is a view of a further embodiment of the brace of the present invention including two crossing additional strut members for additional support of the ATFL;

FIG. 9 is a view of one of the strut members of FIG. 8;

FIG. 10 is a perspective view of an adjustable embodiment of the right ankle brace i of the present invention with a C-Hook closure mechanism;

FIG. 11 is a detail of the closure mechanism of the brace of FIG. 10;

FIG. 12 is a top view of the ankle brace of FIG. 10;

FIG. 13 is a side view of the posterior side of the ankle brace of FIG. 10;

FIG. 14 is a view looking down from the top of the leg portion of the ankle brace of FIG. 10 illustrating the opening of the brace of FIG. 10;

FIG. 15 is an additional view looking down from the top of the leg portion of the ankle brace of FIG. 10 illustrating the opening of the brace of FIG. 10;

FIG. 16 is a side lateral view of the right ankle brace in accordance with the adjustable embodiment of the present invention illustrating straps for adjustability and using Velcro closure patches;

FIG. 17 is side lateral view showing the details of two other closure mechanism of the right ankle brace illustrating anchor and receptacle latching closure mechanisms;

FIG. 18 is a detail of a posterior/lateral view of the right ankle brace in accordance with the adjustable embodiment of the present invention illustrating a slide groove and rod closure mechanism;

FIG. 19 is side lateral view showing the details of a latch and cable closure mechanism of the right ankle brace in accordance with the adjustable embodiment of the present invention;

FIG. 20 is front view of the right ankle brace in accordance with a further embodiment of the present invention;

FIG. 21 is a lateral side view of the right ankle brace of the embodiment shown in FIG. 20;

FIG. 22 is medial side view of the right ankle brace of FIG. 20;

FIG. 23 is a lateral side view of the right ankle brace of FIG. 20 with a different tensioning mechanism;

FIG. 24 is a lateral side view of the right ankle brace of FIG. 20 with another tensioning mechanism;

FIG. 25 is a detail of the rotating tensioning mechanism of FIG. 24;

FIG. 26 is a lateral side view of the right ankle of FIG. 20 with another tensioning device;

FIG. 27 is side view of a further embodiment of the lateral side of the right ankle brace in accordance with the present invention having pockets for additional stiffening members;

FIG. 28 is a side view of the medial side of the ankle brace of FIG. 27;

FIG. 29 is a side view of the lateral side of the stiffening inserts of FIG. 27;

FIG. 30 is a side view of the medial side of the stiffening inserts of FIG. 27;

FIG. 31 is a view looking back from the anterior of the foot portion of the ankle brace of FIG. 27;

FIG. 32 is a view looking forward from the posterior of the foot portion of the ankle brace of FIG. 27 and

FIG. 33 is view from the front edge of the stiffening inserts of the ankle brace of FIG. 27.

FIG. 34 is a view of the lateral side of a brace having an integral shin guard.

FIG. 35 is a front perspective view of a further embodiment of the adjustable brace of the present invention having first closure means;

FIG. 36 is a view of the brace of FIG. 35 having second closure means and without the distal strap,

FIG. 37 is a view of the further embodiment shown in FIGS. 35-39 with the closure straps open toward the rear;

FIG. 38 is a view of the brace of FIG. 36 with a third closure means;

FIG. 39 is a view of the brace of FIG. 36 with a fourth closure means;

FIG. 40 is a top view of a closure mechanism assembly in accordance with another aspect of the present invention;

FIG. 41 is a top view of a strap and male member of the closure mechanism of FIG. 40;

FIG. 42 is a side perspective view of the female receptacle of the closure mechanism of FIG. 40;

FIG. 43 is a cross section of the closure mechanism of FIG. 40 showing the male member as it is being press fit into the closed position in the female receptacle;

FIG. 44 is a cross section of the closure mechanism of 40 showing the male member after s press fit into the closed position;

FIG. 45 is a side view of the male closure member of FIG. 40;

FIG. 46 is a side end view of the closure member assembly of FIG. 40,

FIG. 47 is a top view of a first web of interconnected receptacles of the present invention which form a single row;

FIG. 48 is a top view of a second web of interconnected receptacles of the present invention which form a matrix;

FIG. 49 is a top side view of the weld of interconnected receptacles of FIG. 49;

FIG. 50 is an illustration of the first and second receptacle webs in position in a joint brace in accordance with the present invention;

FIG. 51 is an illustration of the joint brace of FIG. 50 including a top laminate layer to reinforce the area including the receptacle webs;

FIG. 52 is a detail of a further embodiment of the receptacle web of the present invention;

FIG. 53 is a side view of the second embodiment of the closure mechanism of the present invention shown in FIG. 52;

FIG. 54 is a cross-section of FIG. 53; and

FIG. 55 is a top view of a closure mechanism of FIG. 52.

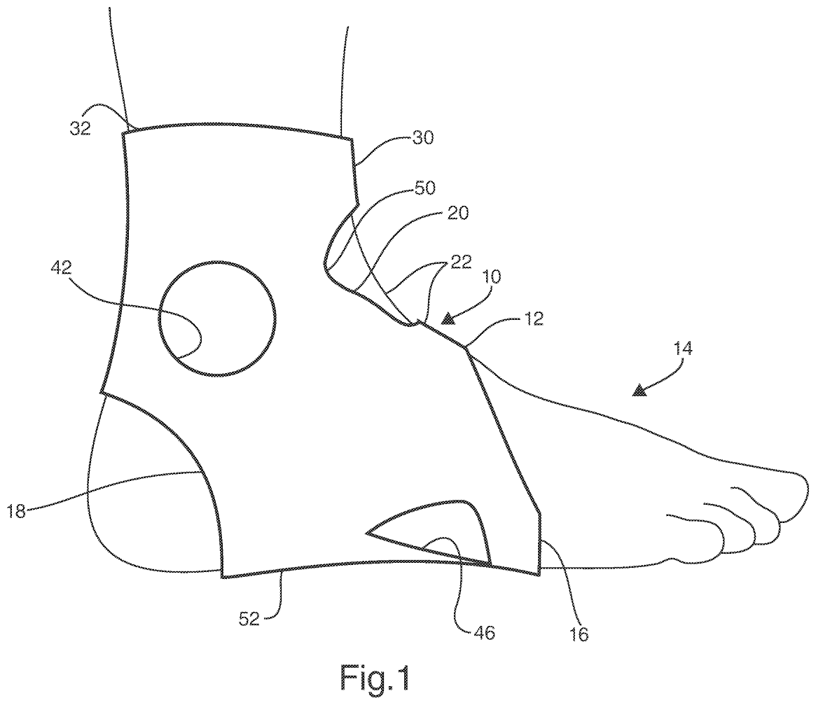

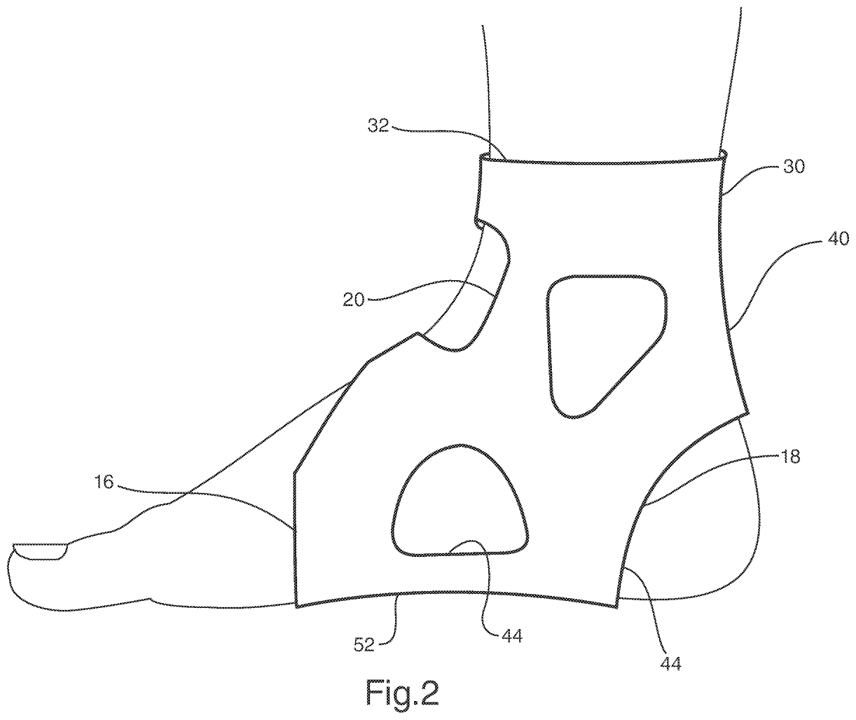

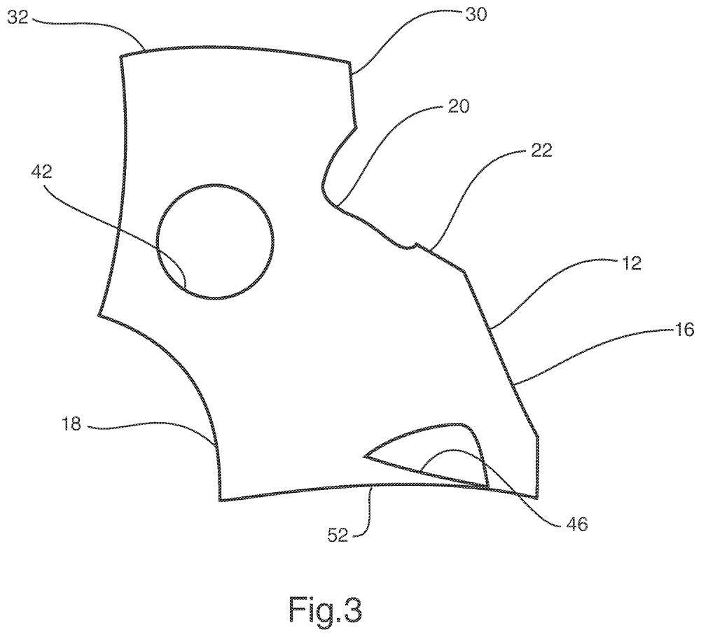

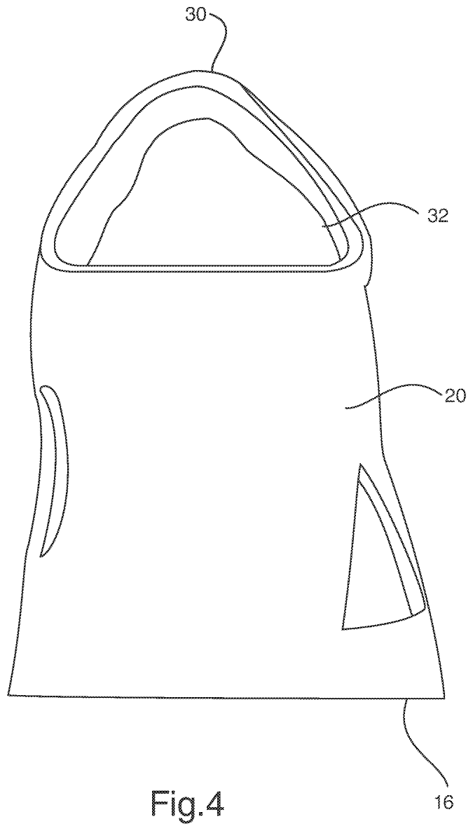

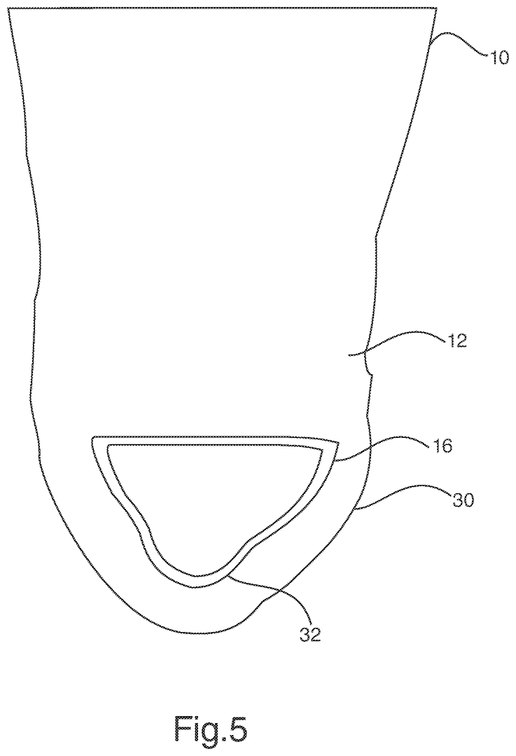

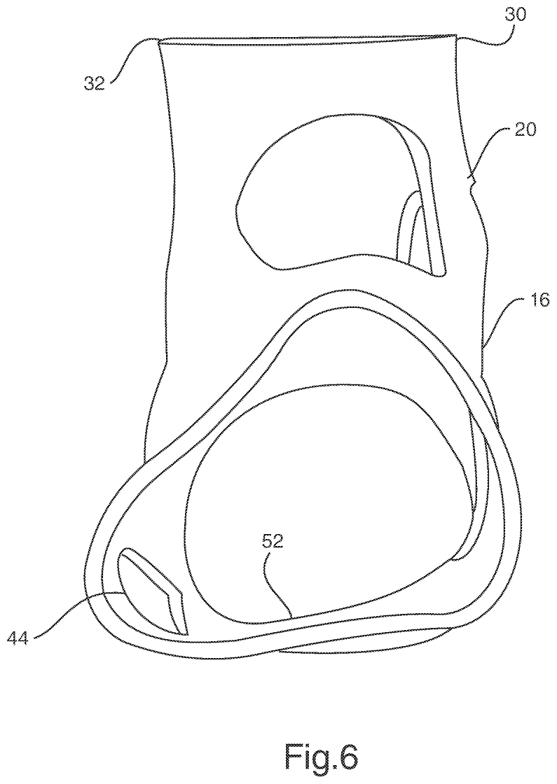

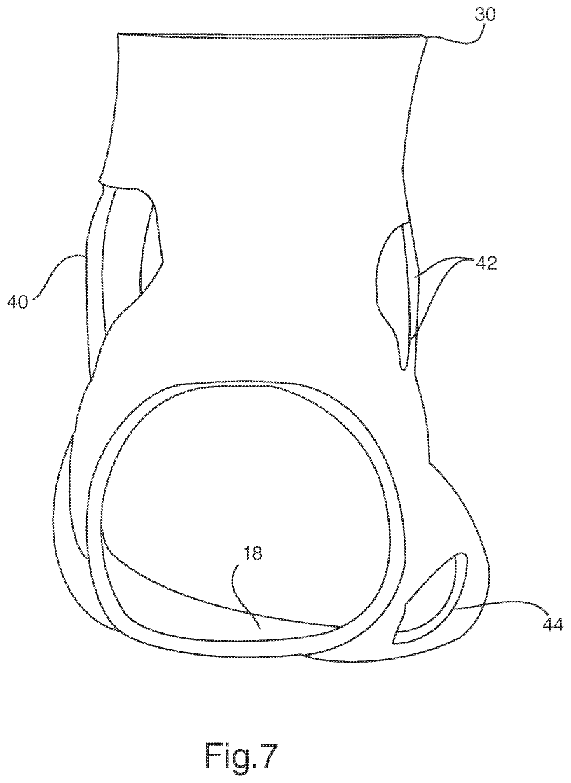

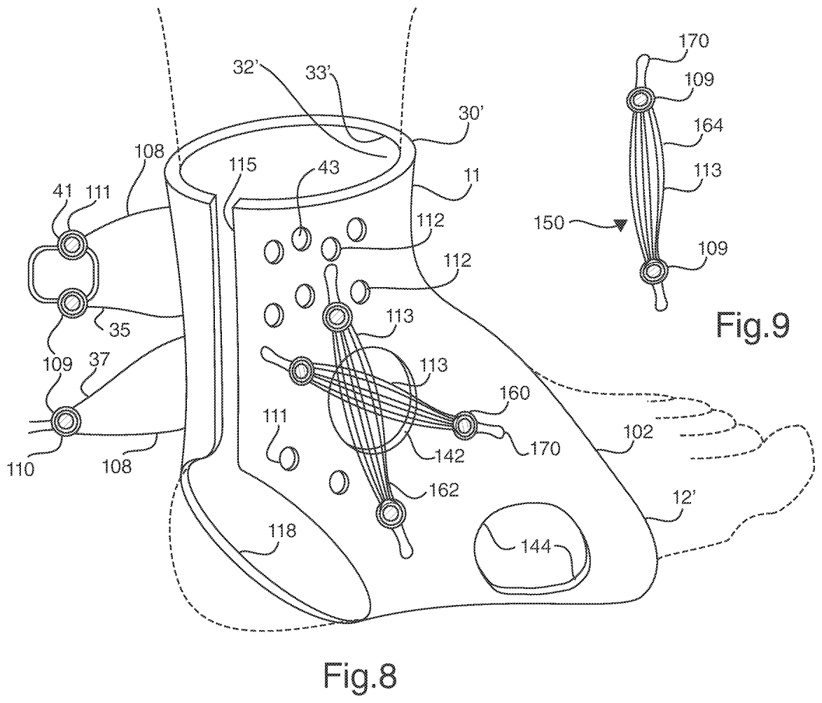

### DETAILED DESCRIPTION OF THE INVENTION

In the ankle brace 10 that is shown, a more distal portion 12 encircles the read-foot of the user 14. The brace is provided in a right version and a left version which are mirror images of each other, and also can be provided in multiple sizes, including for example small and large, or pediatric, ladies and men. The brace is illustrated as a right ankle brace and the left ankle brace is a mirror image of the right ankle brace shown.

The brace is made of a web of flat elastomeric compound or material, which, if opened, would form a flat sheet of relatively uniform or uniform thickness and comprised of a homogenous composition, which optionally includes reinforcing material such as fiber, but which is preferably not a mesh, woven or non-woven fabric in this configuration. The foot portion 12 has a distal opening 16 that is configured to snugly surround the user's foot, at approximately the neck of the fifth metatarsal through the plantar surface to the middle of the first metatarsal and arching proximally toward the tibial fibular talar joint over the dorsal surface of the foot. At the other end, the foot portion 12 ends on the plantar side posterior to the end of the medial arch in a heel opening 18 suitable to expose the fat pad of the heel (approximately ½ of the way posterior toward the heel end of the calcaneus) on the posterior side, and below the insertion of the gastrocnemius into the Achilles tendon as it extends upward on the leg to form the bottom boundary of the leg portion of the brace. The foot portion 12 ends on the anterior side of the ankle in an opening 20 at the “eye of the ankle”, i.e., on the superficial aspect of the anterior ankle at the joint of the tibia/fibula/talus (or the “TFT” joint). The foot portion 12 includes a web of material 22 (preferably molded or cast) that covers an area corresponding to the cuneiforms and the cuboid bone and the navicular bone. On the medial and lateral sides of the brace, the foot portion runs diagonally between the anterior and posterior openings where it loins the leg portion 30 which surrounds the bottom portion of the leg or the vertical portion of the ankle approximately ⅓ of the way up the lower leg, and below the bellies of the distal aspect of the gastrocnemius.

The leg portion 30 of the brace includes a proximal opening 32 that encircles the lower leg sufficiently above the lateral and medial malleoli in order to provide a suitable anchor on the leg of the user for the forces applied by and to the brace. This portion of the brace also forms a continuous loop.

In addition to the previously described openings including the two terminal openings there are several other functional negative areas or “fenestrations” (used herein to mean areas of decreased resistance, including for example through openings, as well as areas in which there are material changes, such as a more stretchy or less cross-linked or even a thinner web of material) in the brace. The two terminal openings include the first or distal most 16, having an edge that runs across the mid-foot on the plantar side, and arching back toward the TFT joint over the top of the foot to the proximal aspect of the metatarsals; and the second or proximal most opening 32 forming a roughly circular opening which encircles the lower leg about 1.5-3.5, and preferably 2-2.5 inches above the malleoli or a third of the way up the lower leg and below the belly of the gastrocnemius.

The functional openings include the opening at the heel 18, which is open to or excludes coverage of a significant portion (i.e. 75% or more or all of) of the surface area of the heel pad, having an edge just in front of the medial process of the calcaneal tuberosity, and on the leg portion of the brace just above the insertion of the Achilles Tendon superior to the calcaneal tuberosity. Further, the brace includes two openings that correspond 1) to the medial malleolus 40 and 2) to the lateral malleoli 42. On the medial side, the malleolus opening 40 is roughly trapezoidal in shape bounded at the top with a roughly horizontal straight edge that is slightly wider than the malleolus and with parallel straight anterior and posterior edges in which the posterior edge is just long enough to accommodate the length of the malleolus and the anterior edge extends below that border to about the insertion of the tibionavicular ligament in the sustentaculum tali of the calcaneus. On the lateral side, the malleolus opening 42 is more elliptical or oval, is slightly smaller than on the medial side in part because the medial malleoli tends to be smaller, and in part in order to retain greater resistance and anatomical support and this opening approximates a size slightly larger than an average size of a malleolus. In addition, there is a roughly triangular (preferably an isosceles with equal sides or even more preferably, an equilateral triangle having a rounded superior angle) opening 44 on the medial side of the foot portion at the juncture of the plantar covering of the foot portion with the mid-foot covering and in an area corresponding to the juncture of the navicular and the first cuneiform as the opening extends upward on the foot at the rear of the medial arch. The lateral side of the foot portion of the brace also includes an opening 46 at the boundary of the plantar covering which again is roughly triangular, but more advantageously, an isosceles triangle with the equal sides extending 1) along the edge of the plantar covering, and 2) from the upper most angle (i.e. toward the dorsal aspect of the mid-foot) backward to the posterior most angle. The opening ends proximally in front of the tuberosity of the fifth metatarsal, and distally roughly 0.5-1 inch behind the terminal opening of the foot portion at the neck of the fifth metatarsal.

The brace includes the foot portion 12 and the leg portion 30 which seamlessly join together at the ankle joint to form one integrated service and is comprised of an interrupted web of elastomeric material in which the interruptions or openings together with the material characteristics of the material define the manner in which the brace functions. In particular, the sleeves forming the foot portion 12 and the leg portion 30 are separated by the heel opening 18 and the TFT opening 20 which allow the foot portion and the heel portion to form a joint 50 there between, and to accommodate movement at the ankle joint without unnecessary material or bunching. This is a particular advantage for a soft brace that is worn underneath a shoe, other athletic footwear, or a shin guard. In addition, the combination of the malleoli openings 40,42 and the two openings at the medial and lateral surfaces 44,46 of the plantar covering 52 of the foot portion of the brace act so as to provide direction as to resistance of force sustained within the web of material that is defined by the combination of the openings. Thus, the brace of the current invention is designed to allow as much safe freedom of movement to the wearer as possible, but to provide resistance to movement that could be harmful. In particular, the device is intended to inhibit inversion in plantar flexion (and to help stabilize the syndesmotic ligament) so as to avoid “rolling” an ankle. The brace is intended to provide external support tantamount to external ligaments and or fascia, which reinforces in proper places, but which relieves pressure where it is needed. Thus, the device acts in tension and compression to buttress the syndesmotic ligament at the top, and in the cross-configuration to buttress the ATFL (anterior tibiofibular ligament), and the CFL (calcaneal fibular ligament), with a medial web member that buttresses the deltoid ligament. In addition, the elastomeric nature of the brace material, coupled with the form can act to provide energy re-balance to the wearer, where the kinetic energy created in a muscular exertion of the user is re-circulated or re-coiled to the user, while inhibiting potentially dangerous forces applied to the joint complex. The “spring” that results, and the resilient contact of the brace with the surface of the ankle also provides a proprioceptive feel to the user that helps to protect the ankle joint.

It is a further advantage in some instances to provide the brace with supplemental tensioning means that can be adjusted to suit a particular user. This embodiment is also illustrates a rear opening aspect of the invention in which the leg portion has a slit 115 between the medial and lateral portions that preferably overlap or abut each other to encircle the leg, and from which straps 108 extends to allow for closing, tightening or tensioning, and which also include closure means, in this illustrations pucks 109, that pop into holes or grommet reinforced openings to retain the medial and lateral portions in the continuous looped arrangement. It is of advantage that the tensioning means act to inhibit stress to the syndesmotic ligament, to the TFTL, and to the TCL. Thus, the tensioning means advantageously extend from the plantar covering (or optional footplate) diagonally upward across the anterior hinge of the ankle in the vicinity of the cuboid and navicular bones, and possibly even to a further tensioning member or anchor at the proximal end of the lower leg portion of the brace. These means can include straps, laces or cable members that are designed so as to provide for adjustable degrees of tensioning, as well as adjustable directions of tensioning to allow the wearer to customize the feel and size. One advantageous closure is a watch strap type closure with a pulley at sinus tarsi level to retain the tensioning bands in an anatomical position, and having tensioning posts protected with hinged door on a button at the fibula for extra security. Also, the tensioning mechanism can include a winding mechanism that translates the rotation of a tensioning dial member to the tensioning strap in order to increase the tension provided by the tensioning strap. These means can include straps, laces or cable members that are designed so as to provide for adjustable degrees of tensioning, as well as adjustable directions of tensioning to allow the wearer to customize the feel and size.

In a specific example of this embodiment, the brace of the present invention includes one or more, and preferably two, optional external add-on supports, which are designed to oppose a detrimental loading of the joint in directions, could prove harmful to the ligaments of the joint involved. Thus, this brace includes the foot portion 12′ and the leg portion 30′ of elastomeric material forming sleeves separated by the heel opening 18′ and the TFT opening. In this version, the lateral malleoli opening 142 has at least one supplemental support 150 that acts to resist the line of force that typically occurs when a person rolls their ankle. More particularly, the support 150 comprises a pair of strap members 160, 162 that cross the malleoli opening and fasten at either side of the opening so as to form an X across the opening. Each strap includes a middle section 113 provided in a dimension (here including a wider intermediate portion 164) and of a material to give the desired resistance, and having attachment means external to the middle section, which are shown as puck members 109 that can be popped into a retained engagement with retaining recesses or “grommets” that are provided in the ankle brace member. The straps 160,162 also include extensions 170 that allow the user to apply a force to the puck 109 to dislodge them from the retaining recesses 112. Thus, the brace of the current invention is designed to allow as much safe freedom of movement to the wearer as possible, but to provide resistance to movement that could be harmful. In particular, the device is intended to inhibit inversion in plantar flexion (and to help stabilize the syndesmotic ligament) so as to avoid “rolling” an ankle. The brace is intended to provide external support tantamount to external ligaments, and in this case to the ATFL, and or fascia, which reinforces in proper places, but which relieves pressure where it is needed. Thus, the device acts in tension and compression to buttress the syndesmotic ligament at the top, and in the cross-configuration to buttress the ATFL (anterior tibiofibular ligament), and the CFL (calcaneal fibular ligament), with a medial web member that buttresses the deltoid ligament.

The present invention is designed to provide some syndesmosis stability above the malleoli. In a further adjustable embodiment, it illustrated with a rear entry, i.e. open toward the posterior portion of the leg, but with an adjustable closure fixation point more anterior or anterolateral, (preferably not medial), with tension from posteromedial to lateral so as to pull the fibula anteriorly to help with syndesmosis stability and ankle. The optional superior band is comprised of a reasonably high tensile strength to protect the syndesmosis. The brace is designed to provide a definite end to plantar flexion and inversion and also some level of protection on the syndesmosis.

It is envisioned that the supports could be struts built into the lateral side of the brace on the lateral side of the brace posterior to the malleolus and extending between the foot portion and the superior portion of the leg support. These struts should have a definite endpoint at say 90-110% of physiological plantar flexion/inversion before easing to a firm stop at which point there is recoil. The basic sleeve of the brace is intended to be very tight on the user with a low tensile strength and durometer so that it molds well to the ankle. The struts have a high tensile strength that eases to a firm endpoint before recoiling. This is advantageously accomplished by providing elements (for example such as one or more fibers, cable or bands that are optionally sinusoidally placed) that have a high resistance to stretch embedded within or carried on the elastomeric sleeve member. This brace acts in tension rather as a buttress as in the prior art.

In addition, tensioning or closure mechanisms permit the wearer to pull through them and get a feel of tension, which provides a reassuring feel to the wearer. This tension is set such that it could result in a very high tensile strength at the end of range of range so that it can be really quite stiff within a range that is totally sale for the user. Optional closure mechanisms include various mechanisms, such as Velcro, watch strap level backs closure, hook and eye, pin and post, buttons, zippers, cables, laces to name a few. One advantageous closure is a watch strap type closure with a pulley at sinus tarsi level to retain the tensioning bands in an anatomical position, and having tensioning posts protected with a hinged door on a button at the fibula for extra security. Also, the tensioning mechanism can include a winding mechanism that translates the rotation of a tensioning dial member to the tensioning strap in order to increase the tension provided by the tensioning strap.

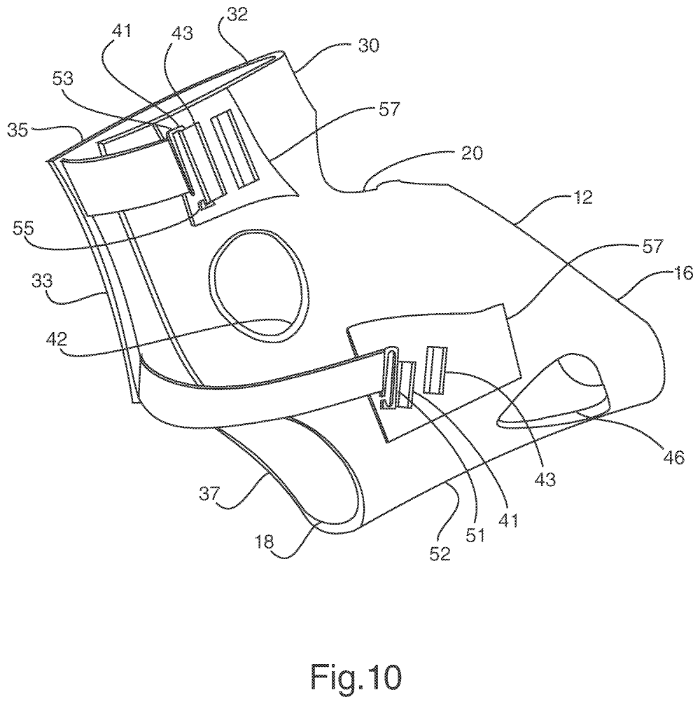

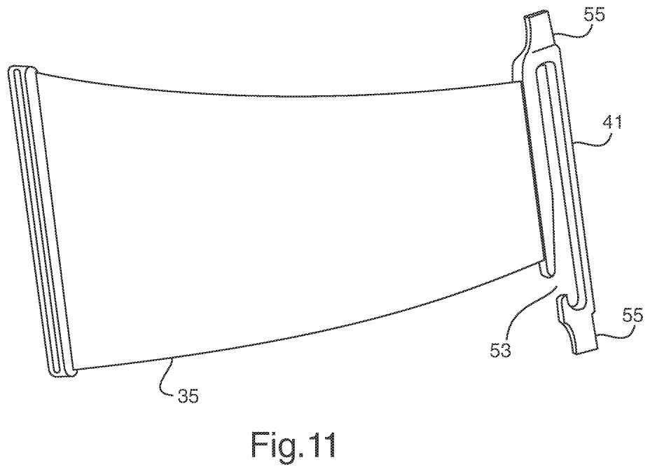

In a further adjustable or closable embodiment of the invention, the leg portion 30′ of the brace includes a proximal opening 32′ that encircles the lower leg sufficiently above the lateral and medial malleoli in order to provide a suitable anchor on the leg of the user. This portion of the brace is open to form a planar web 33′ (i.e., a flat band) that can be closed to form a continuous loop about the lower leg. The web 33 includes one or more extension 35 that can be a strap or band of varying thickness and which is of a length preferably so that the leg portion 30′ fully encircles the lower leg and that the strap, band or cable 35 that extends from a first side of the web 33 can be pulled to a desired tension and secured by means of closure means 41 on the strap or medial side to mating closure means on the lateral side of the web 33. The embodiment shown in FIG. 10 includes an upper strap 35 and a lower strap 37. Various closure mechanisms can be used at this juncture, including straps, bands, webs, and cables having a closure means 41 that mates with a corresponding closure means 43 on the lateral side of the sleeve.

The superior band 35 is integral with the top (i.e., the superior edge) of the leg portion 30″ of the brace and at least in part, defines the size and shape of the proximal opening 32″ that encircles the lower leg. The superior band 35″ includes a closure mechanism that mates with a member on the band or on the brace body or on an attachment or strap on the brace to allow the closure of the brace, as well as sizing and tensioning as is desirable. Various closure mechanisms are illustrated herein. The brace also includes a posterior band 37″ which closes the rear of the leg portion 30″ closer to the ground and which can overlap from the medial to lateral side as shown in FIG. 10 or from the lateral to medial side as shown in FIG. 35. The posterior band 37″ also includes a closure mechanism which cooperates with a mating member on the brace.

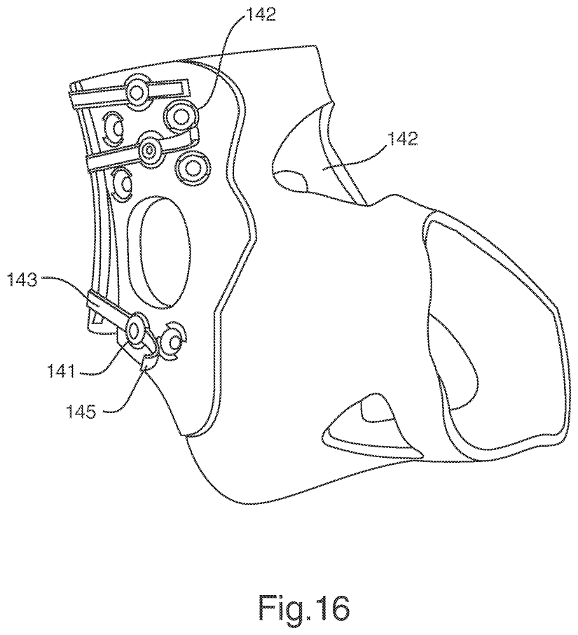

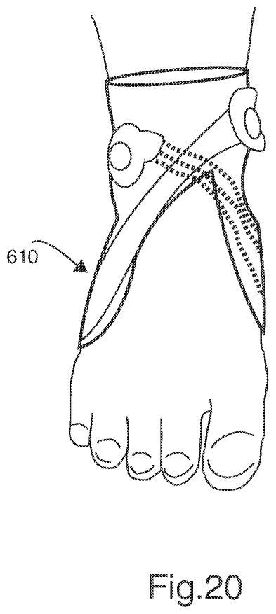

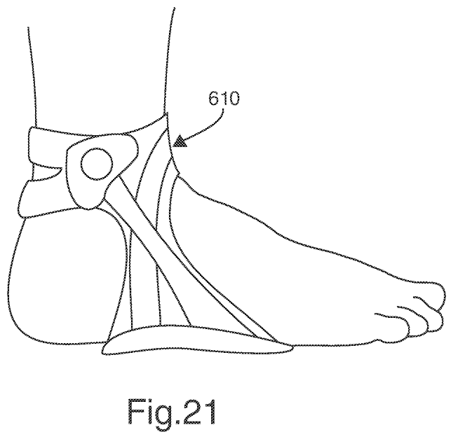

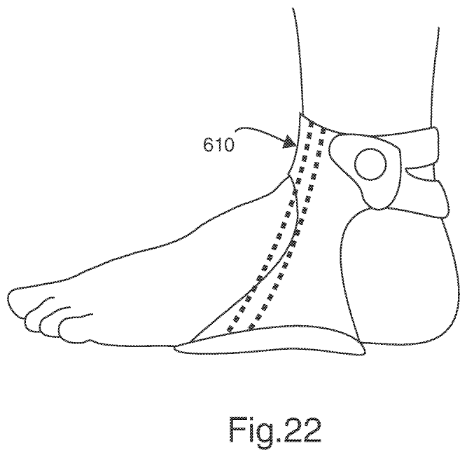

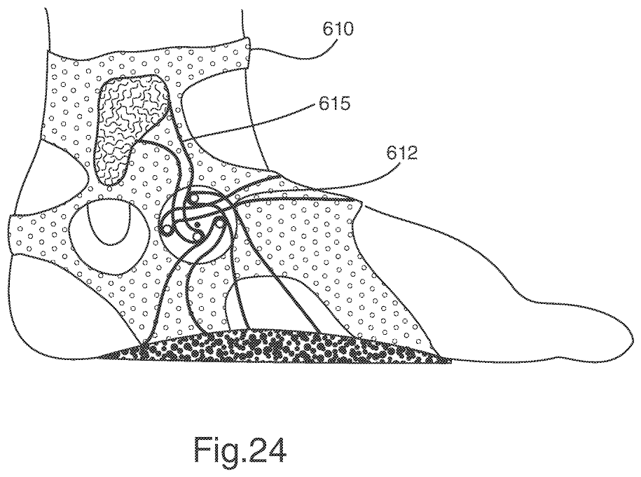

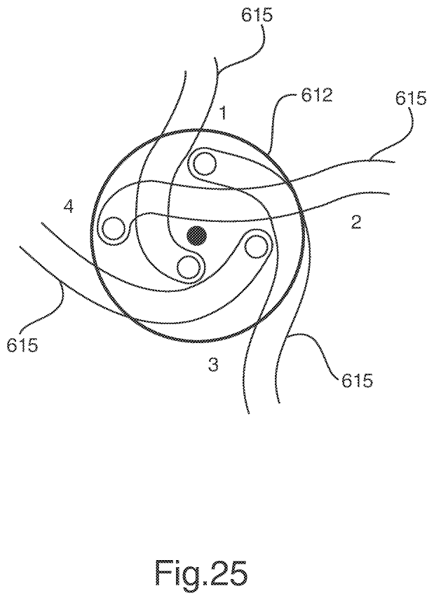

The adjustable embodiment is also illustrated in FIGS. 16-26 with multiple types of lateral closure members or tensioning mechanisms that are circumferentially aligned so that the strap is drawn into a tighter, or more highly tensioned engagement, but in this case, preserving the direction of pull. For example, the closure means 41 is a C-hook having a closed front edge 51 that provides for the line of engagement with the loop closure means 43 on the lateral edge. The C-hook also has a top tab 55 and a bottom tab 55 that provide the user with a handle for hooking the C-hook into the loop. The area in the lateral sections 57 that have the lateral closure means can be reinforced by a laminate of material or a stiffer or more durable material. The straps can project directly from the medial edge or otherwise be attached to it, and also preferably include a taper so that they are wider at the origin and taper down to the insertion et the C-clasp. They are preferably 1-2 mm, and more preferably about 1.5 mm thick, and could be removable, such as by tongue-n-groove, for example with the top strap sliding in from the top into a containment groove in the medial edge and from the bottom into a containment groove in the medial edge.

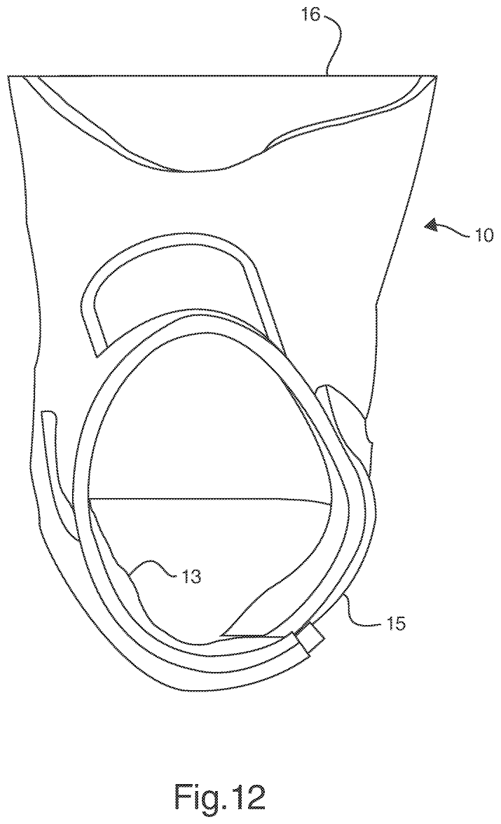

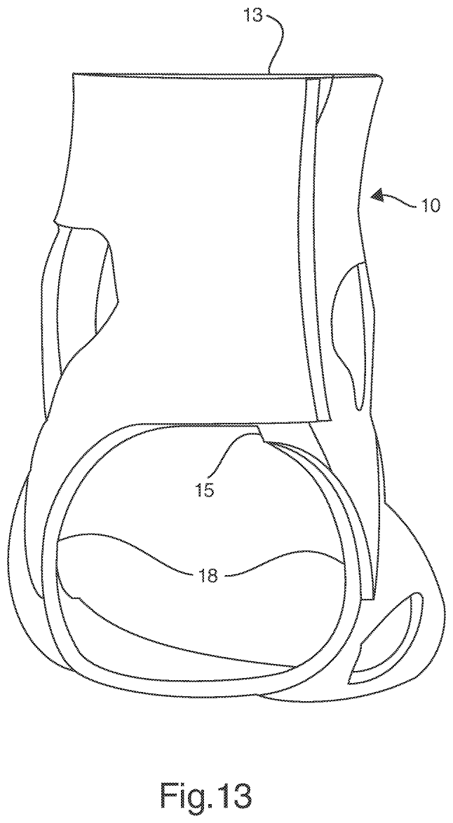

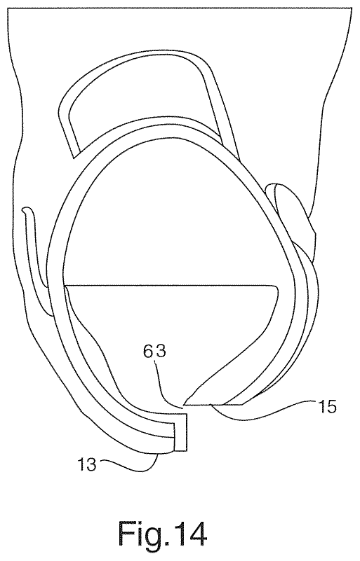

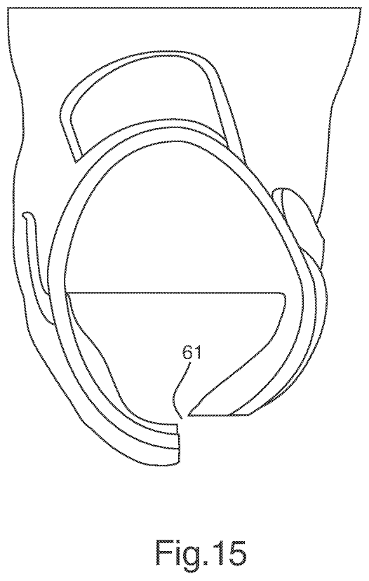

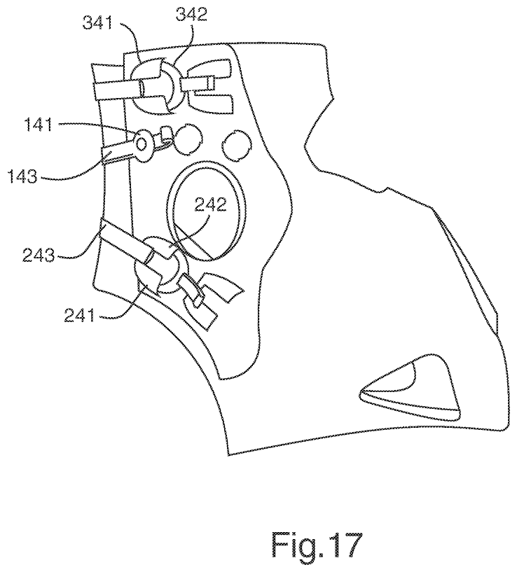

FIG. 12 illustrates a top view of the brace in a closed position and FIG. 13 illustrates a rear view of the overlap of the medial side 13 with the lateral side 15 of the brace 10. FIG. 15 illustrates the leg portion of the brace prior to and in FIG. 17 after the tensioning of the sleeve. In FIG. 14, it can be seen that the opening slit 61 (and in FIG. 15 after tensioning at 63) starts at 6:00 o′clock and extends laterally. This avoids the overlap occurring directly posterior to the Achilles tendon, which could cause aggravation with running, jumping or use of the ankle joint over time. The medial flap of the leg portion is pulled counterclockwise toward the lateral flap which tends to thin the material out slightly (depending on the Poisson's ratio) and resulting in less thickness and a lower profile. Optimally the overlap in the sulcus with the lateral flap is at about 4:30-5:00 o′clock (relative to the anterior medial line). The lateral flap is thinned down near the slit to 1.5-2+/−0.5 mm in thickness. The strap and material are designed so that a short distance pull creates a relatively large amount of stress. The placement of the mating lateral closure members is thus dictated on the configuration and material choices so that multiple locations close together result in a wide selection of resulting tensions and sizes and allowing the user a significant range of tension within a small range of pull.

FIG. 15 illustrates the sleeve post tensioning, and wherein the pulling takes the posterior flap to 5:00 o′clock. This allows about 90° of workable circumference for placement of the lateral closure means. Preferably, the closure means are low profile and will not aggravate the user. The bands coming off the medial flap can be relatively short and thus result in good tensioning reproducibility and also for a significant amount of tensioning with a relatively stiff elastomer. Preferably the bands originate from a hard polymer, which is embedded into the end of the sleeve to provide increased durability and a more even pull. The bands can taper in height, taller at the origin, and thinner at the insertion (i.e., the location of the male closure) to further distribute the stress. There is optionally a pull connected to the male closure member, such as a stiff polymer or cloth which acts as a handle for the user during assembly and which will lay flat when the closure means is assembled.

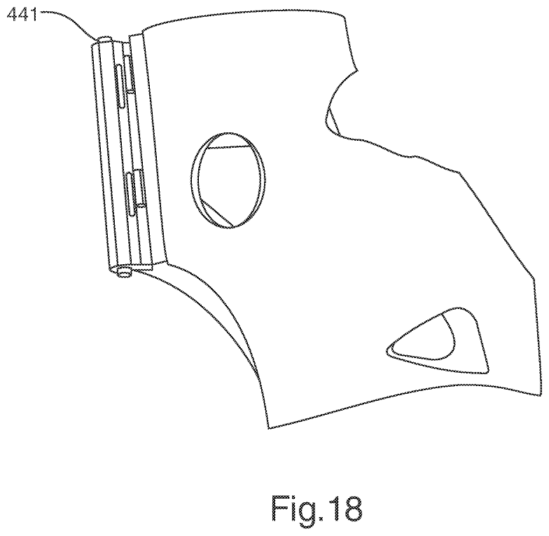

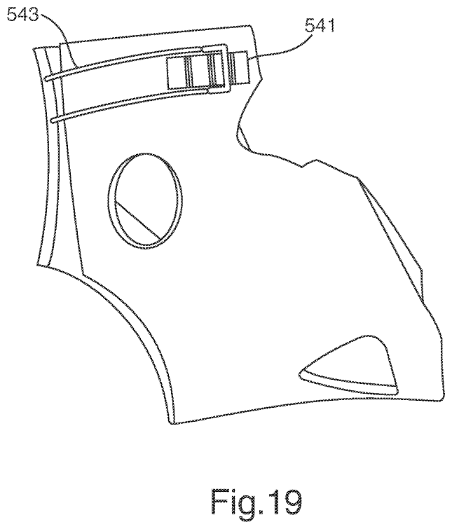

FIG. 16 is a side lateral view of the right ankle brace including multi-band configuration with Velcro patches 141 attached to thin straps 143 and having multiple areas of mating hook and loop closure means that allow both a variation in the distance of tension and according also the amount of tension applied to the medial side of the brace. The straps each also include a tab 145 that permits the straps to be pulled around the leg and fastened to the desired mating patch 142. FIG. 17 is side lateral view showing the details of two other anchor and receptacle closure mechanism of the right ankle brace which include tabs 242, 342 and female receptacles 24,341 also attached to straps 243. FIG. 18 is a side lateral detail of the right ankle brace including a full flap closure 441 i.e., a groove and anchor closure. FIG. 19 is a side lateral view showing another closure mechanism of the right ankle brace, which is a latching mechanism 541 with cables 543 for tensioning.

Also the brace may be provided as an adjustable brace with a rear entry and closure means that allow sizing of the open portion. For example, it is advantageous to provide an open loop for the leg portion which can be tailored to a desired size, and which can even be re tailored at a later point. The anatomic location of the closure mechanism is important, and ideally, this is at the posterolateral aspect of the ankle joint; housed between the Achilles and distal fibula. This minimizes the interference with many athletes' functions as well as minimizes general interference incurred during gait, again, depending on the mechanism of choice. Alternatively, the closure mechanism can be located on the medial side with the tensioning means (or straps) pulling in the direction of the struts laterally. Advantageously, the tensioning means provides for ½ to 1 centimeters of adjustability, (in particular if the brace is provided in three sizes), depending on the material of the tensioning means and the size range for which the brace is intended.

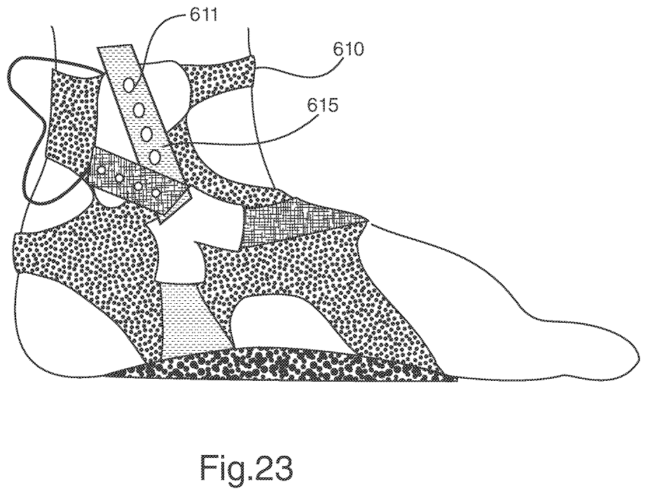

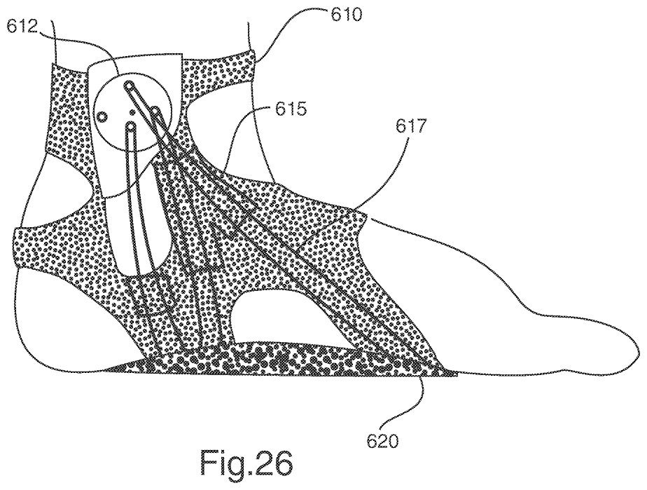

FIGS. 20-22 illustrate an ankle brace 610 of the present invention, which includes these specific additional tensioning means to support the TFL and CFL. Further embodiments of this invention are shown in FIGS. 23-26 in which support is specifically provided at this anatomical landmarks, and also in which tensioning means including a watch strap type button 611 or rotating wheel 612 both attached to tensioning straps 615 are provided to tightened the tension as desired. FIG. 26 illustrates a version having a Y-shaped sleeve in sinus tarsi to maintain an ideal cross-over with elastic cords 617 that glide within the mechanism and having a foot plate 620 and tunnels within the brace 610 to maintain the position of the straps 615.

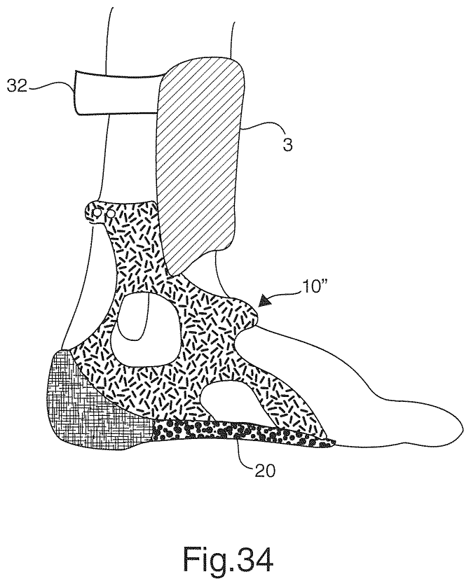

FIG. 34 illustrates a brace in accordance with the present invention having an integral shin guard. The brace 10″ of the present invention can also include a more resilient or stiffer integral shin guard or pad 3 which is formed as a part of the brace, either by molding and changing the material characteristics or by adhering the shin pad to the brace. The shin guard can include a strap 32, which also encircles the leg, but higher than the leg portion 30″, and which acts to secure the guard in place during use.

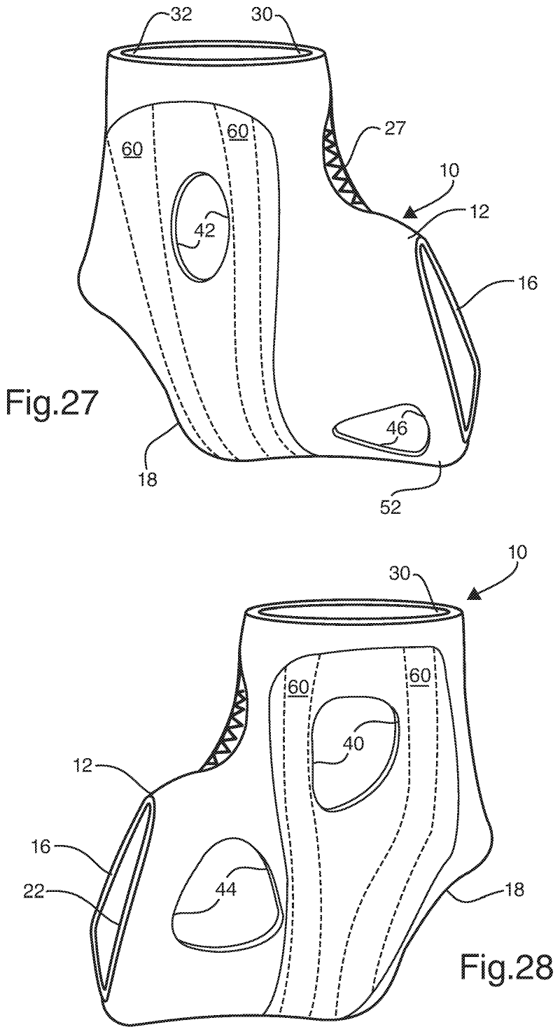

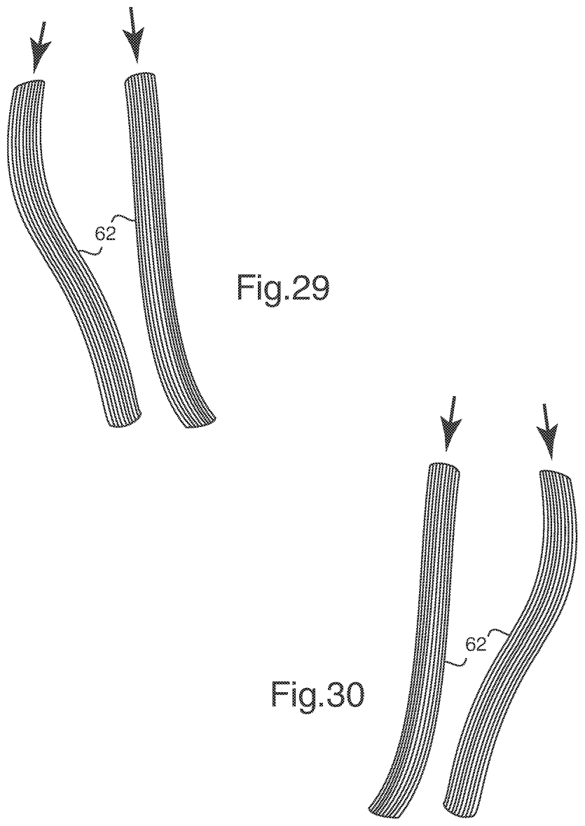

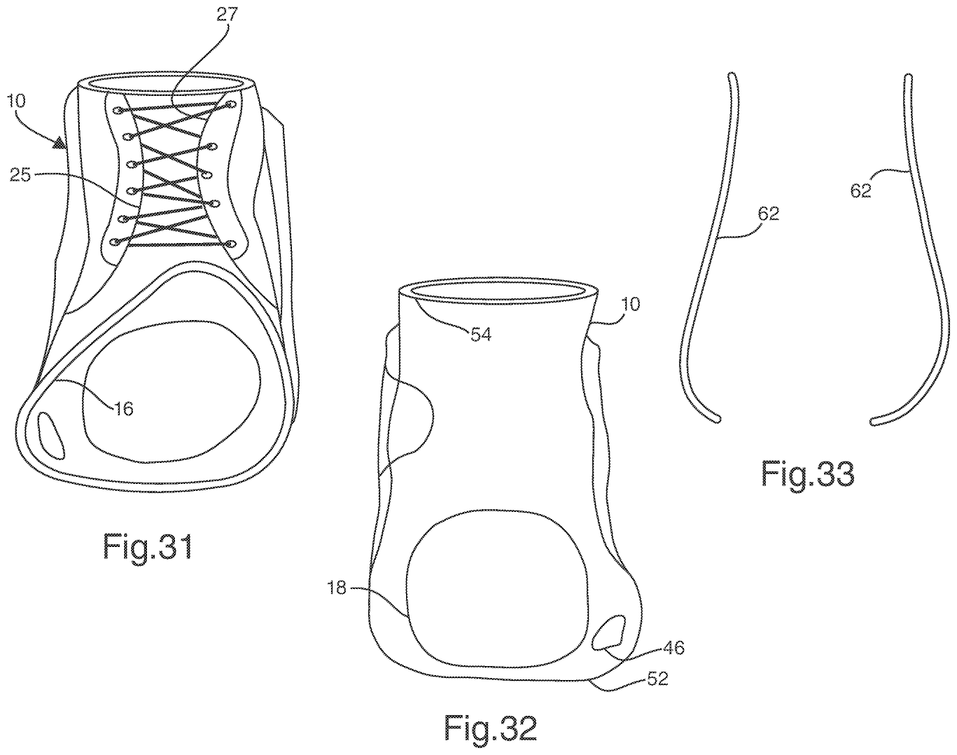

In the brace shown in FIGS. 27-34, the brace includes a leg portion 30 and a foot portion 12 having a proximal opening 32 and distal opening 16 and an intermediate tarsal opening 27 and malleoli opening 42, 44. In addition, the brace includes pockets 60 which include stiffening supports 62 which are housed in channels within the pockets.

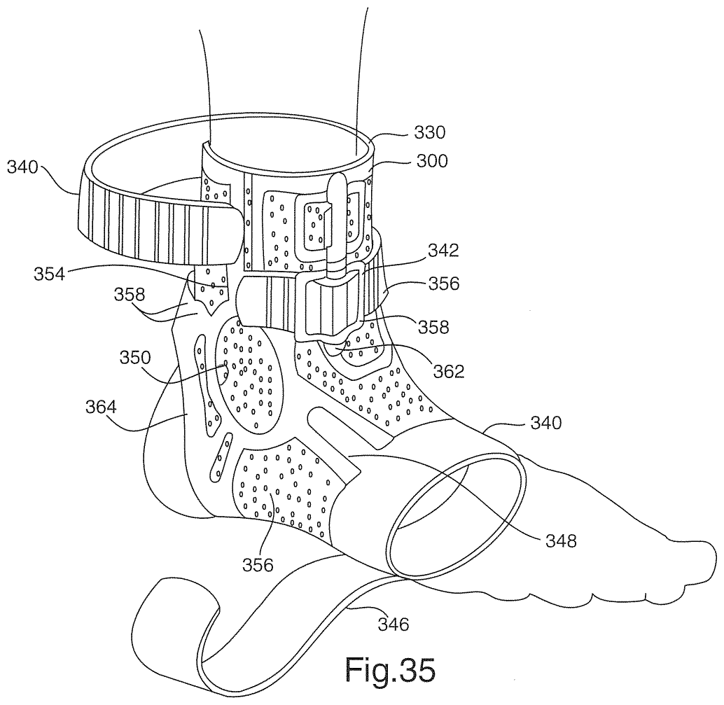

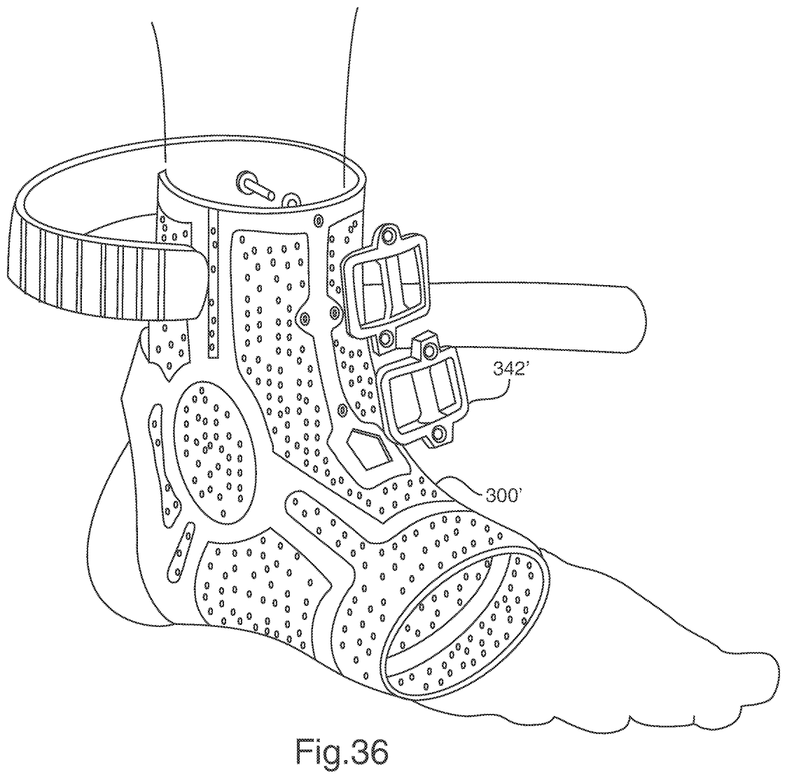

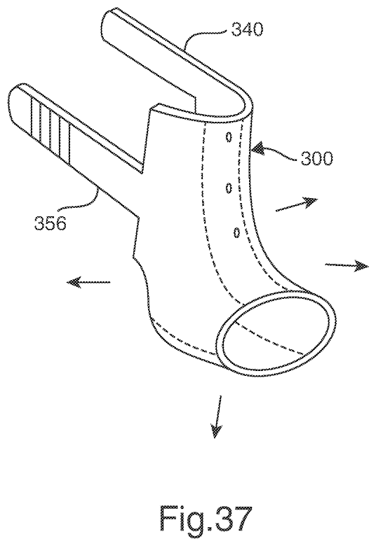

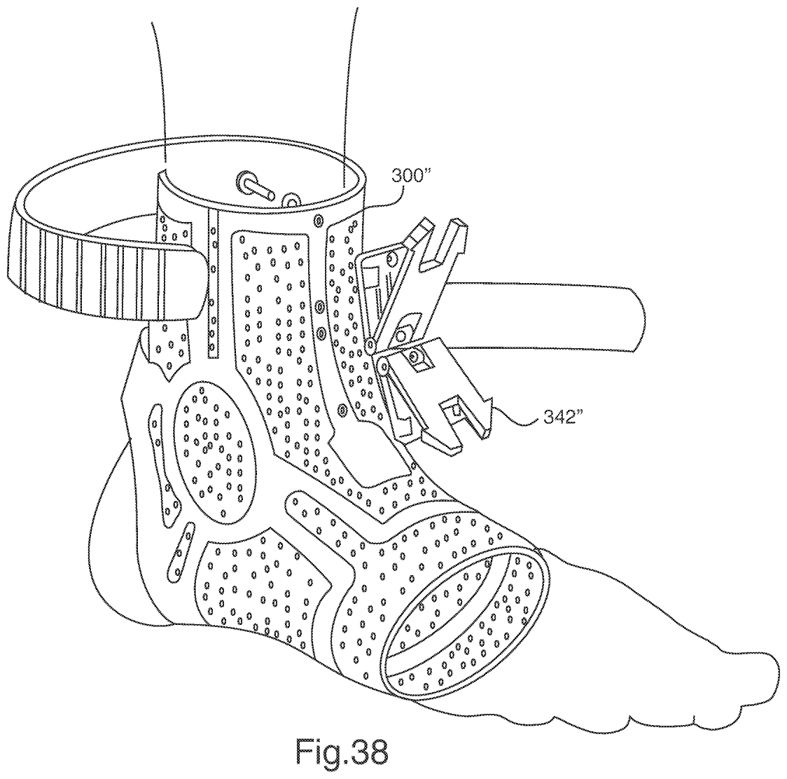

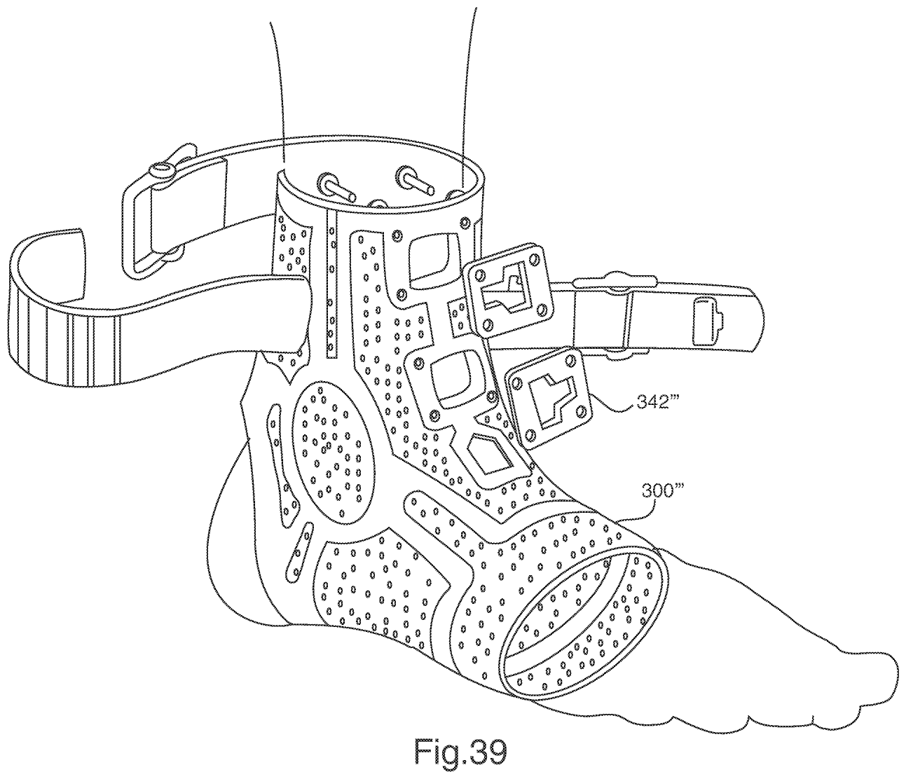

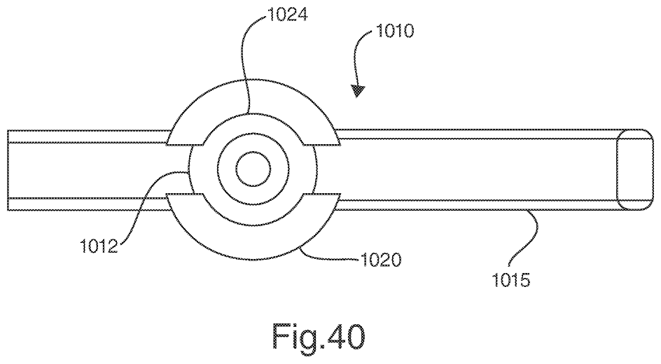

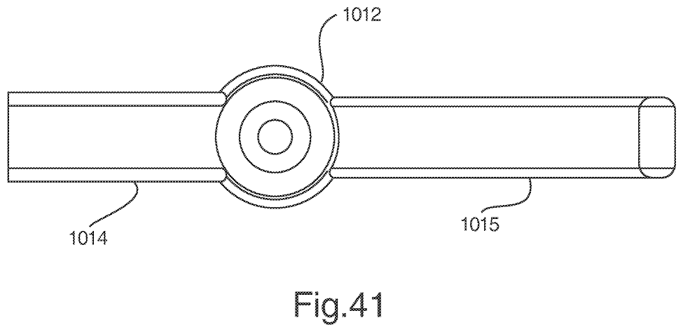

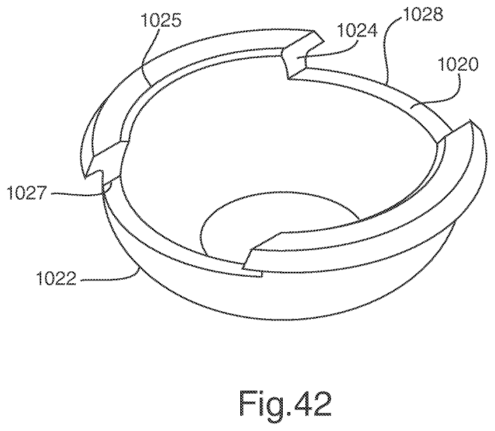

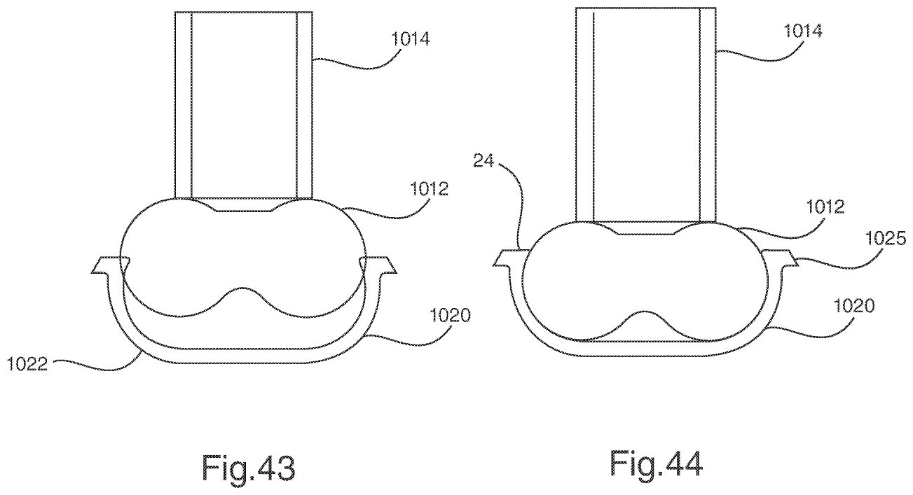

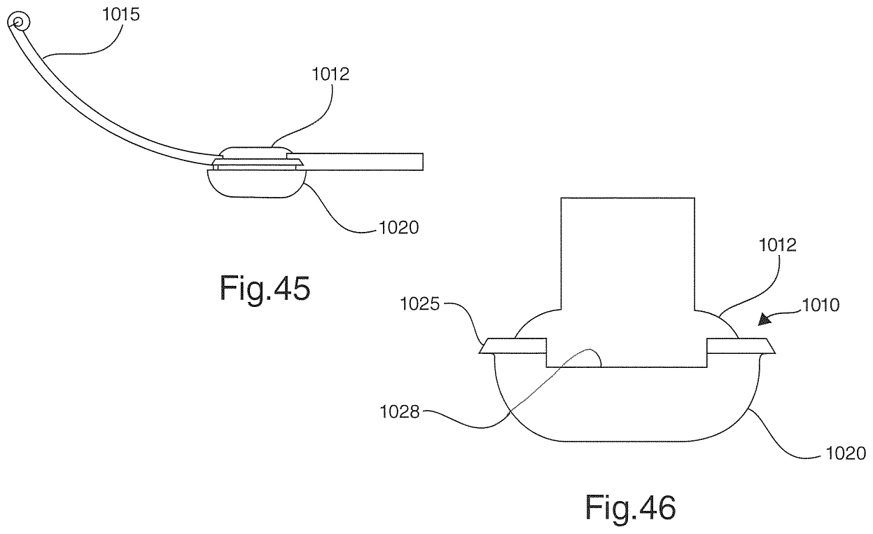

In addition, in a further embodiment, the brace is illustrated as including a framework 300 of a stiffer (i.e., higher durometer material of approximately 95 durometers +/−15, preferably +/−10 and most preferably +/−5 on the Shore A scale. This framework 300 includes a proximal anchor 330, which encircles the upper leg and, in this case, includes a strap 340 which engages a hard plastic closure mechanism 342 on the front of the brace. The proximal anchor is a band 346 and forms a flat continuous (i.e. looping back on itself) ring of relatively narrow width and constant thickness and which circles the foot. Advantageously, the ring 346 also includes at least one, but optionally more, (i.e. two three, four or more), V-shaped (or other shape which include a wider opening and a tapering portion which resists but will allow for expansion of the circumference of the ring) gusset 348 which allows the proximal anchor to expand without losing its function as an anchor in order to allow for size variations of the wearer. The framework also includes an opening 350, 352 on each of the lateral and the medial sides, preferably oval as previously described, to accommodate the malleoli. Struts extend from the proximal anchor to the malleoli openings. Further struts 354 extend upward from the malleoli openings to the proximal anchor to complete the circuit between the distal anchor and the proximal anchor. This version also includes a lower strap 356, which wraps the ankle at a lower position and from the lateral to the medial side and fastens with a buckle 358 on the front of the brace. The framework is also shown with a front support 362 that forms a base for the buckles, and a rear member 364 frames the heel opening and is linked to the rest of the brace through links 358 to the malleoli openings. The fenestrations in this case, are actually areas of integrated softer material, for example having a durometer of 35+/−10, and preferably +/−5 on the Shore A scale. This material is a relatively soft sheet of elastomeric material, with a uniform thickness from surface to surface, which is slightly sticky to the touch, as can be formed by injection molding or by casting at a lower cross-linking. This softer portion of the sleeve can also include perforations to allow for perspiration or can include texturing to the surface for proprioceptive reasons. FIGS. 36, 38 and 39 illustrate variations of the sleeve without a distal strap, and having different forms of a closure mechanism 342, 342′ 342″. FIG. 37 shows the brace open to the rear with the straps extending straight behind the brace 300.

The brace is made, for example by molding such as injection or silicone molding or casting, a bio-compatible elastomer from a material of suitable durometer to provide the desired fit, and elastomeric characteristics. The brace preferably is made of a material that exhibits equal stretch in at least two dimensions (i.e., the X, Y directions). This material can be made more resistant to provide further support, for example of the syndesmotic ligament, by various means, including the additional of supports or struts which might be provided by an integral (same material) thickening of the brace in a defined area, or by changes in the material itself, such as higher rate of cure or cross-linking or the addition of other materials such as reinforcing fibers or the use of a second elastomeric material having greater resistance to an applied force, like a higher durometer or Young's modulus or modulus of elasticity, and which could be embedded in the brace, co-molded, or adhered to the inside or outside of the brace. The brace is designed to allow motion with a limited endpoint; to encourage the recoil of energy and to allow for the potential prevention of harmful forces, i.e., the brace permits motion that is safe within a defined range but inhibits abnormal or dangerous motion.

The brace forms a two-part sleeve which is in substantial contact with the skin of the user between the two terminal ends of the brace. Thus, in the first embodiments, while there are fenestrations or openings in the brace, the remaining web occupies at least 40%, and preferably at least 50%, and even more preferably at least 60% or 75% of the area defined by the outline of the brace. In the embodiments having a more rigid framework and fenestrations with a softer web of material these ratios are reversed. The inferior surface may advantageously include a mesh, surface treatment or textured finish to increase the breathability and to prevent slippage.

The following represents some dimensions with respect the cutouts or fenestrations:
arc-length of lateral malleolus cut-out=78.5 mm arc-length of medial malleolus cut-out=120.7 mm;
arc-length of base of 5th met cut-out=82.8 mm;
arc-length of ant ankle cut-out=141.7 mm; and
arc-length of navicular prominence cut-out=108.2 mm.

Some of the minimum distances between cut-outs (approximately) are as follows:
lateral malleolus cut-out & ant ankle cut-out=28.6 mm;
medial malleolus cut-out & ant ankle cut-out=15.8 mm; and
medial malleolus cut-out & navicular cut-out=20.0 mm.

In addition, the material is intended for a particular tactile experience at the surface of the skin of the wearer so as to provide a proprioceptive reminder to the wearer of the type that has been found to help inhibit ankle sprains. It is preferable that the brace has a slightly tacky feel at the skin interface. Thus, the brace provides biofeedback to alert the stabilized joint so that it acts to inhibit undesired motion within that joint. In further embodiments, the brace may be put on wet, or over an inner sleeve that helps to enhance the tactile experience, such as including a roughly textured surface having a pattern of bumps, ridges, dimples, cross-hatching, or protrusions.

The brace of the present invention can be used in a variety of joints. While the present invention can be used for hinged joints it is preferably for use in joint complexes, so that for example the “ankle” brace actually is intended to stabilize the ankle, subtalar and talonavicular joints, and the concepts set forth herein can be useful in support of other joints, including for example those located at the wrist, the elbow, the shoulder, the knee, and the fingers.

The present invention also has application for treatment of plantar fasciitis, medial and lateral (elbow) epicondylitis, toe and finger/thumb synovitis. A particular advantage of the present invention is that the brace is designed to “stretch” up to a defined endpoint and that an effective “stop” is reached by the a tensioning member that acts like an elastomeric “ligament” placement. This can be provided by a change in structure of the brace, such as increased volume of material designed to limit the stretch, a different material characteristics, such as a higher degree of cross-linking, or change in material including for example, a cohered portion along a lateral edge, or an adhered portion along a top or bottom surface (including fabric which could be woven, and which could serve additional purposes, such as skin interface, bacterial or fungal control or odor control), or embedded materials, such as fibers or wires which exhibit relatively little stretch and are configured to provide a limit to a range of stretch at a given stop point.

The invention relates generally to a molded elastomeric sleeve of a biocompatible material having a defined hardness and elasticity, shape, and configuration in three dimensions (adapted to the anatomy of a hypothetical user). For the ankle this means a brace configured to end on the foot at the neck of the fifth metatarsal and on the lower leg below the belly of the gastrocnemius, and having an opening at the heel cup and at the eye of the ankle joint, at the medial and lateral malleoli, and at the navicular bone and optionally including additional support of additional material or a stronger or less elastomeric material on the lateral side which resists a force applied to the ankle in inversion, including, for example, an integral support or attachment such as a tension strap positioned anterolaterally to simulate the direction of the ATFL for more anterolateral stability where there is a support for syndesmotic stability, and one for ankle stability.

The basic sleeve of the brace is intended to be very tight on the user with a low tensile strength and durometer so that it molds well to the ankle. The material of the brace is ideally an elastomer, including for example, a thermoplastic elastomer having a Shore A hardness of 2-50 at 10 sec when measured in accordance with ASTM D2240, and a tensile break at stretch of 2-6 MPa at 23° C. using Die C2 hour when measured in accordance with ASTM D412, tensile stress of 0.08 to 0.8 MPa at strain 100% and 0.2 to 1.5 MPa at 300% at 23° C. using Die C2 hour when measured in accordance with ASTM D412, and an Elongation at break of 800-1200% at 23° C. using Die C2 hour when measured in accordance with ASTM D414, a tear strength of 7.5-20 kN/m when measured in accordance with ASTM D624, and a compression set of 5-30% at 23° C. and at Time 79200 sec when measured in accordance with ASTM D395. Thermoplastic elastomers are suitable materials, with material sold under the trademarks Versaflex CL30, and CL2000X from PolyOne being preferable materials, alone, or compounded with additional materials, such as other cross-linking agents, additional elastomers to achieve material characteristics, reinforcing fibers and fillers, antimicrobial agents, colorants, and fragrances.

The brace in accordance with the invention can include struts laminated or adhered to the outer or inner surface or embedded within the sleeve member, and which have a high tensile strength that eases to a firm endpoint before recoiling. This is advantageously accomplished by providing elements (for example such as one or more fibers, cable or bands that are optionally sinusoidally placed) that have a high resistance to stretch embedded within or carried on the elastomeric sleeve member. This brace acts in tension rather as a buttress as in the prior art. The brace could further include a fabric backing over an entire surface or over portions of surface in order to control the directions of resistance including a weave such as a bias weave fabric, which limits the stretch to one axis and inhibits the stretch along the other two axes.

The through thickness of the sleeve will depend on the material and elasticity but is preferably “low profile” meaning that it can be worn, optionally with socks, under a user's pre-owned shoe, meaning that it does not require a different size than is worn without the brace. Preferably the thickness would be from 2 to 10 mm, with about 5-8 mm on the lateral side and such that the brace still fits into the shoe and is cutout to go around the bony eminences. The medial side does not require the same resistance and could be 3-4 mm.

As designed, the brace optionally includes a self-formed (meaning that the foot plate is only loosely defined by an area of increased thickness or hardness, and that the wearer's foot acts to define the shape of the footplate in use) foot plate which contours around the heel more distally around the base of the fifth, so as to improve ST (sustentaculum/talar) joint stability. Alternatively, the footplate could be integral with the remainder of the brace, but could be more definitely defined, for example, by formation of a different, and potentially stiffer, or harder material. Thus, the foot plate could optionally be provided in a different material, for example a harder, or less stretchy material or this could be accomplished using a different configuration. Also, the footplate could optionally be thicker (i.e., by 0.5-2 mm on the lateral side for approximately the length of the foot plate or at least 50% of the length and approximately ⅕ to ⅓ of the width to bias the foot to the outside and in order to promote control the tension on the syndesmotic ligament.

For the embodiment showing an integral shin guard, the leg portion 30″ of the brace includes a proximal opening 32″ that encircles the lower leg sufficiently above the lateral and medial malleoli in order to provide a suitable anchor on the leg of the user. This portion of the brace is open to form a planar web (i.e., a flat band) that can be closed to form a continuous loop about the lower leg. The web includes one or more extension that can be a strap or band of varying thickness and which is of a length preferably so that the leg portion 30″ fully encircles the lower leg and that the strap, band or cable that extends from a first side of the web can be pulled to a desired tension and secured by means of closure means 41 (shown here as a pin and hole, but which could be various other closure mechanisms) on the strap or medial side to mating closure means on the lateral side of the web.

The brace of the present invention includes a more resilient or stiffer integral shin guard or pad 30 which is formed as a part of the brace, either by molding and changing the material characteristics or by adhering the shin pad to the brace. The shin guard can include a strap 35″ which also encircles the leg, but higher than the leg portion 30″, and which acts to secure the guard in place during use.

The brace is intended to last at least one season of intermediate level of non-professional use (i.e. 2-3 times per week), which is based on usage on the idea that the running shoes need to be changed every 300 or so miles, which is approximately 7-8 miles per week in a 9 month soccer season, or alternatively for one month of heavy use, and wherein the limiting factors include the continued support and configuration integrity, odor-free characteristics, and stickiness or tack to provide for the proprioceptive reinforcement.

The present invention also provides a closure mechanism 1010 that includes a male member 1012 in the form of an elastomeric round puck that has an interference fit with a female receptacle 1020 in the form of a round dish, which has at least one edge member 1024 that acts to hold the male member in place in the receptacle. The female member is also elastomeric to allow an opening action as the male member is pressed into it. Also, the retaining edge 1024 can be elastomeric and formed as part of the receptacle, or can be an additional member fit into position, such as a metal grommet member. Preferably, the retaining edge is circumferential, but can include one or more interruptions, so as to accommodate the elongate member, which the male member secures. Thus, for a strap or band 1014, the retaining edge 1024 includes an opening 1028, which is slightly wider than the strap or band, and in addition, the male member may also include a pull tab 1015 which can be used to tension and for pressing the male member into the receptacle. The retaining edge 1024 can include a second interruption 1027 to accommodate the tab as well, which holds it out of the way from interfering with the item that bears the closure mechanism.

The male member comprises a 8 mm diameter×4 mm height round or donut shaped member that can be fabricated from hard poly/nylon/ABS for example or any material ideally suited to inhibit plastic deformation yet allow both an audible and tactile acknowledgement of engagement. Acetal could also be an optimal material. Preferably, the male and female members are not the same material as sleeve, but the female and male members may be fabricated from the same of different materials from each other. Preferably, they have a durometer in range of 70-80 Shore D hardness and may be fabricated of materials including (3100) PC/ABS or (3400 or 3600) Polypropylene. The male member, or puck, is attached to a polymer band which is 5 mm wide×1.5 mm thick at the insertion to the male member. A significant radius is given to the edge on the underside so as to aid in the deformation of the cusp of the female member retaining edge. The same significant fillet is given to the top side of the male member so as to avoid rough or harsh edges and to make it pleasing to touch. In addition, the top and underside of the male member has a concavity to provide for more ergonometric handling.

The male member includes a pull tab opposite the band member, which is 4.5 mm wide×1 mm thick. The pull includes a nub at the end so as to ease gripping and reduce the likelihood of slippage when it is grasped by a user. The pull material is durable and stout, yet easily manipulated so as to lay flat at the final closure or attachment position.

The closure mechanism is comprised of an elastomeric material, which allows the male member to be resiliently held within the female receptacle of the assembly. The material of the closure mechanism can be the same for the puck, dish, and the strap but is preferably not the same material.

The retaining edge may be a self-edge such as is formed by molding or may be an adhered or embedded material. It is ideally embedded into a reinforced section of the item to be closed, so as to provide resistance to the pressure of closure, or to pad the user from feeling it. The maximum diameter of the grommet or retaining edge is 1 cm with an even wail thickness of 0.5 or more. The grommet is currently 4.5 mm in height with 4.0 mm of it being buried within the item that bears it (which leaves 0.5 mm above the surface and where the assembly leaves about 1 mm above the surface of the item, for a low-profile appearance.

The strap member is likely a self-formed strap of the brace member which is ideally an elastomer, including for example, a thermoplastic elastomer having a Shore A hardness of 2-50 at 10 sec when measured in accordance with ASTM D2240, and a tensile break at stretch of 2-6 MPa at 23° C. using Die C2 hour when measured in accordance with ASTM D412, tensile stress of 0.08 to 0.8 MPa at strain 100% and 0.2 to 1.5 MPa at 300% at 23° C. using Die C2 hour when measured in accordance with ASTM D412, and an Elongation at break of 800-1200% at 23° C. using Die C2 hour when measured in accordance with ASTM D414, a tear strength of 7.5-20 kN/m when measured in accordance with ASTM D624, and a compression set of 5-30% at 23° C. and at Time 79200 sec when measured in accordance with ASTM D395. Thermoplastic elastomers are suitable materials, with material sold under the trademarks Versaflex CL30, and CL2000X, from PolyOne being preferable materials, alone, or compounded with additional materials, such as other cross-linking agents, additional elastomers to achieve material characteristics, reinforcing fibers and fillers, antimicrobial agents, colorants, and fragrances.

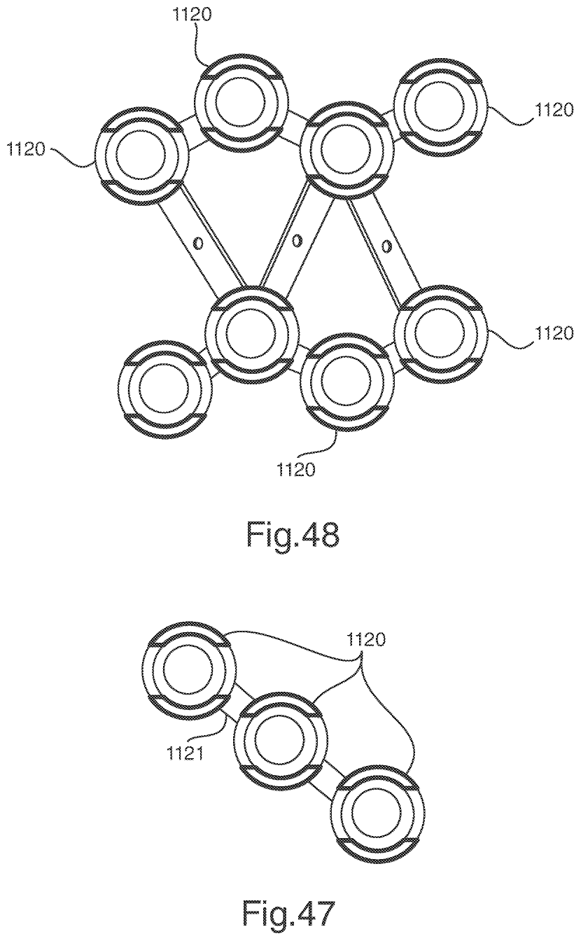

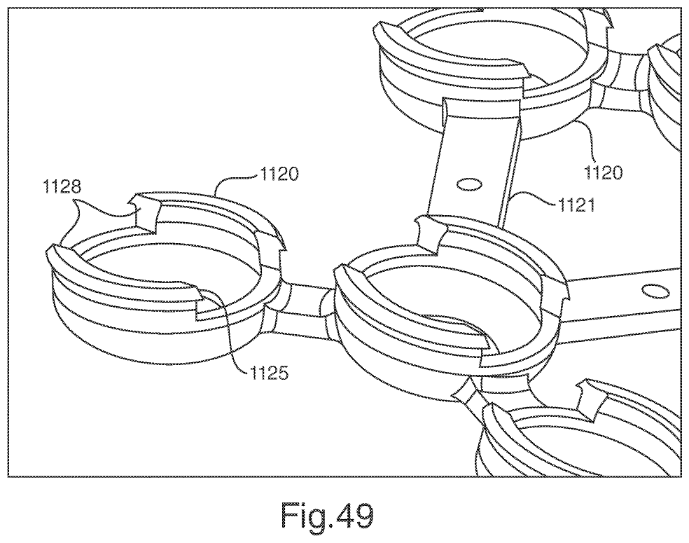

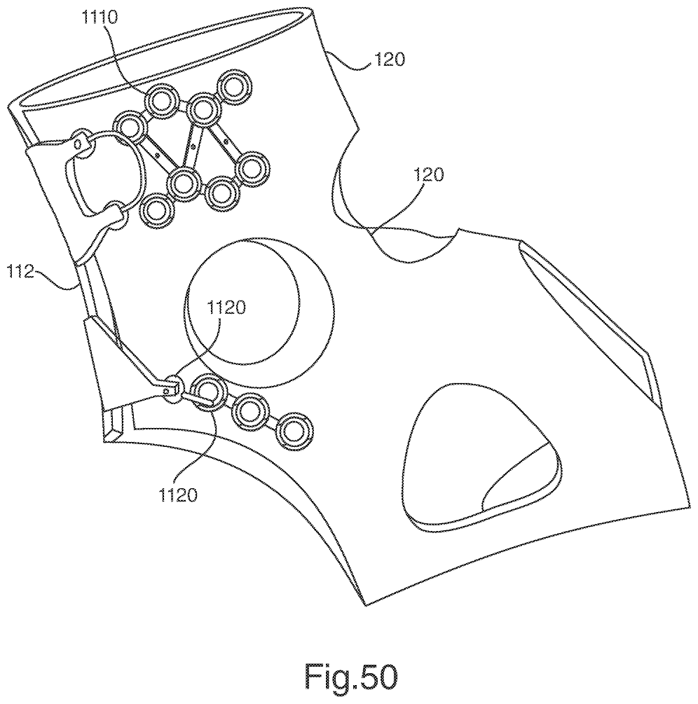

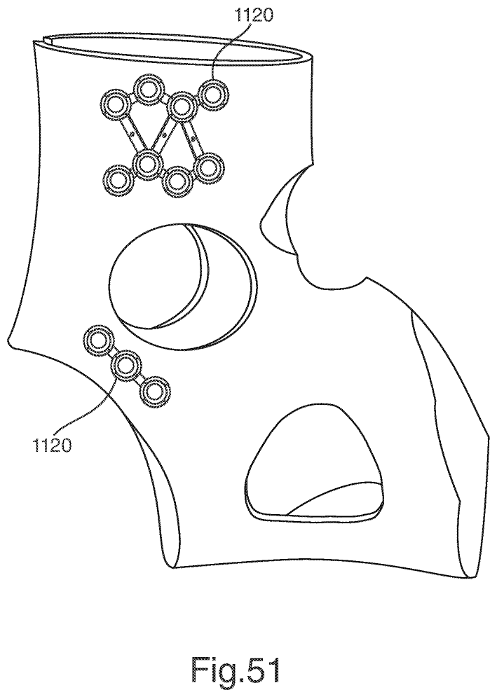

In a further embodiment of the invention illustrated in FIG. 47-FIG. 51 the closure mechanism 1110 includes the male member 1112 connected to a strap of a brace member and to a pull tab for tightening and closing the closure mechanism in the receptacles 1120 which are provided in a series in which lines of linking members 1121 connect individual receptacles 1120 to form a “spray” or a web that has multiple connected receptacles. This configuration reinforces the individual receptacle members and provides for increased resistance to better retain the puck members and to inhibit inadvertent pop-out during use, and also to better retain the receptacles in the brace during the disengagement of the puck members from the receptacles. In a first view shown in FIG. 47, the web forms a line or curve with single members 120 in a row, whereas in a second version shown in FIG. 48, the web comprises two rows of members in order to provide a matrix useful for multidimensional adjustment of the size and/or force applied through the elastomeric material. FIG. 50 illustrates that a laminate can be made in which the web is internal to a top reinforcing layer of material, again to strengthen the resistance against the receptacles from dislodging from the brace. FIG. 49 is a detail of the web of FIG. 48 and FIG. 52 is a detail of the web of. FIG. 47.

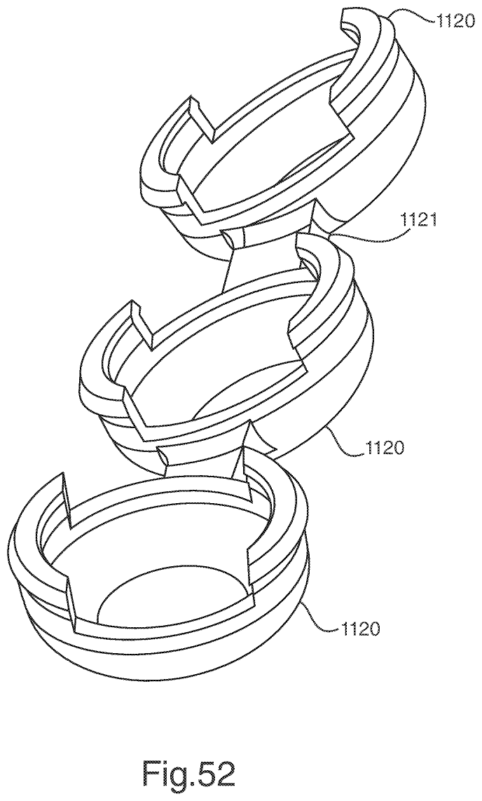

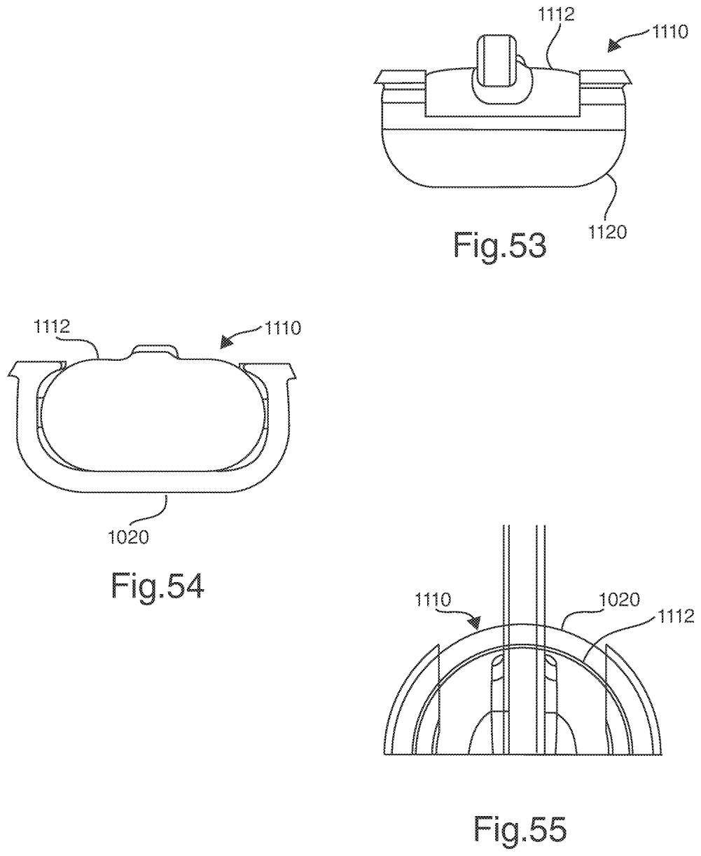

FIGS. 53-55 illustrate an embodiment of the closure mechanism 10′ which is designed for a more robust cooperation between the male member 12′ and the female receptacle 20′. In particular the retaining edge is fuller and more completely engages a top portion of the male member 12′ as can be seen from the cross-sectional view of FIG. 55.

The invention relates to an ankle brace for a hypothetical user having a leg including an ankle joint complex extending from below a gastrocnemius at the proximal side to behind the neck of a fifth metatarsal on the distal side, the brace comprising a sleeve formed from a sheet of biocompatible elastomeric material having a through thickness of from 1 to 7 mm, and the brace has a first portion which is a web formed from the sheet of the biocompatible elastomeric material which forms a first continuous loop about a first axis and a second portion which is a web formed from the sheet of the biocompatible elastomeric material that forms a second continuous loop about a second axis; and the first portion of the web having a proximal end which includes a top opening sized to fit below the belly of the gastrocnemius of the hypothetical user and the distal end of the first portion being connected to the proximal end of the second portion at a conjunction of the first portion and the second portion, and the distal portion of the second portion including a bottom opening sized to fit posterior to the neck of the fifth metatarsal of the hypothetical user; and the web including at least a first fenestration at the conjunction of the first and the second portions which is configured such that the first axis and the second axis are not the same.

It also relates to the previously described ankle brace as set forth above wherein the fenestration is a through hole and wherein the fenestration is an area of decreased resistance in the web and wherein the first portion of the web includes at least one opening for the lateral malleolus and wherein the first portion of the web includes at least one opening for the medial malleolus.

The invention relates to the previously described ankle brace wherein the elastomeric material is a thermoplastic elastomer having a Shore A hardness of 5-95 at 10 sec when measured in accordance with ASTM D2240 or a tensile break at stretch of 2-6 MPa at 23 Die C2 hour when measured in accordance with ASTM D412 or stress of 0.08 to 0.8 MPa at strain 100% and 0.2 to 1.5 MPa at 300% at 23° C. using Die C2 hour when measured in accordance with ASTM D412 or an Elongation at break of 800-1200% at 23° C. using Die C2 hour when measured in accordance with ASTM D414 or a tear at of 7.5-20 kN/m 23° C. and at Time 79200 sec when measured in accordance with ASTM D395 where the elastomeric material could be a elastomeric material is a thermoplastic elastomer sold under the trademarks Versaflex CL30, and CL2000X from PolyOne including such an elastomeric material compounded with an additional materials selected from the group comprising other elastomers, cross-linking agents, reinforcing fibers and fillers, antimicrobial agents, colorants, and fragrances, and in particular wherein the reinforcing fiber is selected from glass, steel and carbon fiber. And this ankle brace could be formed by molding or casting.

The ankle brace also relates to an ankle brace for a hypothetical user having a leg including an ankle joint complex below a gastrocnemius at the proximal side and behind the neck of a fifth metatarsal on the distal side, the brace comprising a sleeve formed of a web of biocompatible elastomeric material having a through thickness of from 1 to 7 mm, and a that has a first portion which forms a first continuous loop about a first axis and a second portion that forms a second continuous loop about a second axis; and the first portion of the web having a proximal end which includes a top opening sized to fit below the belly of the gastrocnemius of the hypothetical user and the distal end of the first portion being connected to the proximal end of the second portion at a conjunction of the first portion and the second portion, and the distal portion of the second portion including a bottom opening sized to fit posterior to the neck of the fifth metatarsal of the hypothetical user; and the web including at least a first fenestration at the conjunction of the first and the second portions and the ankle brace further comprising a removable support member which provides resistance to a force applied to the anterior tibiofibular ligament.

This invention can include the previous ankle brace wherein the removable support member comprises at least one strap that is attached to the sleeve or wherein the removable support member comprises a pair of straps that extend across one another to from an X-shape or wherein the strap is placed in an orientation of an ATFL

The invention also relates to a closure mechanism that include a male member which is attached to an elongate member, and a female member including a recess which accommodates the male member and the female member having a retaining edge which retains the male member within the recess in use.

The invention further relates to the previously described closure mechanism wherein the male member is round, and wherein the male member or the female member is comprised of an elastomeric material and wherein the female member includes a circumferential retaining edge and wherein the elongate member is a strap, band, or cable and wherein the female member includes a circumferential retaining edge and the retaining edge has an interruption for the elongate member and wherein the male member includes pull tab, and wherein the female member includes a circumferential retaining edge and the retaining edge has an interruption for the pull tab.

The invention relates to the previously described closure member wherein the elongate member comprises an elastomeric material is a thermoplastic elastomer having a Shore A hardness of 2-50 at 10 sec when measured in accordance ASTM D2240 and/or wherein the elastomeric material is a thermoplastic elastomer having a tensile break at stretch of 2-6 MPa at 23° C. using Die C2 hour when measured in accordance with ASTM D412 and or wherein the elastomeric material is a thermoplastic elastomer having a tensile stress of 0.08 to 0.8 MPa at strain 100% and 0.2 to 1.5 MPa at 300% at 23° C. using Die C2 hour when measured in accordance with ASTM D412 and/or wherein the elastomeric material is a thermoplastic elastomer having an Elongation at break of 800-1200% at 23° C. using Die C2 hour when measured in accordance with ASTM D414 and or wherein the elastomeric material is a thermoplastic elastomer having, a tear strength of 7.5-20 kN/m when measured in accordance with ASTM D624 and/or wherein the elastomeric material is a thermoplastic elastomer having, and a compression set of 5-30%, at 23° C. and at Time 79200 sec when measured in accordance with ASTM D395. The thermoplastic elastomer could be a thermoplastic elastomer sold under the trademarks Versaflex CL30, and CL2000X from PolyOne and the male member and the female member are formed by molding and the female member could be provided in a series of connected female members and the female members can be connected by linking members.

The invention relates to the previously described closure members arranged in a series to form a line or a curve, including in a series to form a matrix and to a closure mechanism wherein the male member has a shape selected from the group consisting of a button, a donut, a torus, an oval, a sphere, or a cone and wherein the male member, the female member or both for example or a material which inhibits plastic deformation while allowing an audible or tactile acknowledgement of engagement and, wherein the closure mechanism material comprises one or more of polycarbonate, polypropylene, nylon, ABS or Acetal and having a durometer in the range of 70-80 Shore D hardness.

While in accordance with the patent statutes the best mode and preferred embodiment have been set forth, the scope of the invention is not limited thereto, but rather by the scope of the attached claims. Additionally, informal drawings corresponding to the formalized drawings of the present application, and taken from priority application Ser. No. 15/349,617, are contained in the appendix to the specification, which is incorporated herein by reference.

What is claimed is:

1. An ankle brace for a hypothetical user having a leg including an ankle joint complex extending from below a gastrocnemius at the proximal side to behind the neck of a fifth metatarsal on the distal side, the brace comprising a sleeve of biocompatible elastomeric sheet material having a through thickness of from 1 to 7 mm, and a that has a first portion having web of material which is closed by closure means to form a first continuous loop about a first axis and a second portion that forms a second continuous loop about a second axis; and the first portion of the web having a proximal end which includes a top opening sized to fit below the belly of the gastrocnemius of the hypothetical user and the distal end of the first portion being connected to the proximal end of the second portion at a conjunction of the first portion and the second portion, and the distal portion of the second portion including a bottom opening sized to fit posterior to the neck of the fifth metatarsal of the hypothetical user.

2. An ankle brace as set forth in claim 1, wherein the web has a first edge and a second edge and a discontinuity there between and the web is configured so that the discontinuity is not directly over an Achilles tendon of the hypothetical user.

3. An ankle brace as set forth in claim 1, wherein the closure means is attached to a band, strap, cable or tab which extends from the medial or lateral side of the brace and includes one or more of Velcro, C-hooks, pin and post, zipper, tongue and groove, buttons, latch and hook, anchor and anchor receptacle, and laces.

4. An ankle brace as set forth in claim 1, wherein the fenestration is a through hole.

5. An ankle brace as set forth in claim 1, wherein the fenestration is an area of decreased resistance in the web.

6. An ankle brace as set forth in claim 1, wherein the first portion of the web includes at least one opening for the lateral malleolus.

7. An ankle brace as set forth in claim 1, wherein the first portion of the web includes at least one opening for the medial malleolus.

8. An ankle brace as set forth in claim 1, wherein the elastomeric material is a thermoplastic elastomer having a Shore A hardness of 2 to 50 at 10 sec when measured in accordance with ASTM D2240.

9. An ankle brace as set forth in claim 1, wherein the elastomeric material is a thermoplastic elastomer having a tensile break at stretch of 2 to 2 MPa at 23° C. using Die C2 hour when measured in accordance with ASTM D412.

10. An ankle brace as set forth in claim 1, wherein the elastomeric material is a thermoplastic elastomer having a tensile stress of 0.08 to 0.8 MPa at strain 100% and 0.2 to 2.5 MPa at 300% at 23° C. using Die C2 hour when measured in accordance with ASTM D412.

11. An ankle brace as set forth in claim 1, wherein the elastomeric material is a thermoplastic elastomer having an Elongation at break of 500 to 1200% at 23° C. using Die C2 hour when measured in accordance with ASTM D414.

12. An ankle brace as set forth in claim 1, wherein the elastomeric material is a thermoplastic elastomer having a tear strength of 7.5 to 20 kN/m when measured in accordance with ASTM D624.

13. An ankle brace as set forth in claim 1, wherein the elastomeric material is a thermoplastic elastomer having, and a compression set of 5 to 30% at 23° C. and at Time 79200 sec when measured in accordance with ASTM D395.

14. An ankle brace as set forth in claim 1, wherein the elastomeric material is a thermoplastic elastomer sold under the trademarks Versaflex CL30, and CL2000X from PolyOne F-115 A/B 15 Shore A Polyurethane Elastomer, and E1040AL from Quatumcast Material or commercial equivalents.

15. An ankle brace as set forth in claim 1, wherein the elastomeric material is compounded with an additional material selected from the group comprising other elastomers, cross-linking agents, reinforcing fibers and fillers, antimicrobial agents, colorants, and fragrances.

16. An ankle brace as set forth in claim 15, wherein the reinforcing fiber is selected from glass, steel, and carbon fiber.

17. An ankle brace as set forth in claim 1, wherein brace is formed by molding or casting.

18. An ankle brace as set forth in claim 1, further including a framework which cooperates with the web of the first portion and the web of the second portion and comprises a material of a durometer that is 10 to 90 points higher on the Shore A scale.

19. An ankle brace as set forth in claim 18, wherein the framework includes a first anchor at the proximal area of the first portion and a second anchor at the distal area of the second portion, and the framework includes linking members that cooperate to extend between and connects the first second and the second anchor.

20. An ankle brace as set forth in claim 19, wherein the framework includes linking members that act as stiffening ribs in the anatomic orientation of the lateral collateral ligamentous complex of the joint.
